(19) United States
(12) Patent Application Publication
(10) Pub. No.: US 2025/0035581 A1
(43) Pub. Date: Jan. 30, 2025

(54) MULTI-TARGETED, MODULAR VIRUS SENSING PLATFORM

(71) Applicant: BOARD OF REGENTS, THE UNIVERSITY OF TEXAS SYSTEM, Austin, TX (US)

(72) Inventors: Deji AKINWANDE, Austin, TX (US); Dmitry KIREEV, Austin, TX (US); Neelotpala KUMAR, Austin, TX (US); Dalton TOWERS, Austin, TX (US)

(21) Appl. No.: 18/714,512

(22) PCT Filed: Nov. 30, 2022
(86) PCT No.: PCT/US2022/051427

§ 371 Date: May 29, 2024

Related U.S. Application Data
(60) Provisional application No. 63/284,706, filed on Dec. 1, 2021.

(51) Int. Cl. G01N 27/414; G01N 33/543; G01N 33/569

(52) U.S. Cl. CPC G01N 27/4145; G01N 33/5438; G01N 33/56983; G01N 2333/11; G01N 2333/165

(57) ABSTRACT

A biosensor includes a multiplexed array of electrolyte-gated functionalized field effect transistors (FET). Walls fluidically separate each of the FETs from one another provide wells corresponding to each channel of the FETS. A perimeter wall on the upper surface of the multiplex array surrounding the separating walls and traversing the surface of all FETS in the multiplexed array. The perimeter forms a fluidically tight well traversing multiple ones of the FETs. Each of the FETs includes monolayer of a two dimensional material, such as graphene, as a conducting channel, wherein each conducting channel functionalized according to a predetermined agent.

### CROSS-REFERENCE TO RELATED APPLICATIONS

This application claims the benefit of priority to U.S. Provisional Application No. 63/284,706 filed Dec. 1, 2021, which is hereby incorporated herein by reference in its entirety.

### GOVERNMENT SUPPORT CLAUSE

This invention was made with government support under Grant no. ECCS2033846 awarded by the National Science Foundation. The government has certain rights in the invention.

### FIELD

Embodiments of the present invention relate to a biosensor, specifically a field-effect transistor biosensor having multiple functionalized chambers.

### BACKGROUND

Within the recent COVID-19 outbreak, a lack high quality point-of-care systems that can detect specific biomolecules, such as hostile viruses or bacteria, became evident. The time between hosting the virus and developing the systems, getting tested through established methods, is long, and often crucial. Moreover, often the symptoms of different diseases of viruses (e.g. COVID-19 and Influenza) are similar, making hospital-style testing even more complicated. Therefore, there is a need for a portable biosensor system that is (a) rapid, (b) specific, (c) multi-targeted.

The pathology of upper respiratory viruses has regularly presented challenges to global health care systems and their resources. The emergence of new virus variants that can evade communal immunological memory can be rapidly transmitted through airborne mucosal droplets, often resulting in the emergence of sudden seasonal epidemics or pandemics. Over the last century, the most prominent of these viruses have been variants of influenza (Flu), which have been estimated to be responsible for approximately 400,000 deaths annually1. The emergence of the novel coronavirus SARS-COV-2 (COVID-19) in 2019 introduced a new upper respiratory virus that, as of now (September 2022) has led to at least 6.3 million deaths globally.

The COVID-19 pandemic has highlighted the need for new rapid point of care diagnostic systems for upper respiratory viruses, especially for high population density areas where the transmission can be the most potent and diagnostic availability and turnaround time the most limited. Significant challenges in respiratory diagnostics include the establishment of assays with a limit of detection (LoD) suitable for identifying early infections, minimizing false positive rates, and reducing the time to perform the assay. The current standard, the reverse transcription polymerase chain reaction (RT-PCR) isn't ideal for identifying early respiratory infections, as demonstrated by the United States' Center of Disease Control's (CDC) recommendation that these assays should be performed 5 days after an exposure to ensure maximal viral titera. Additionally, RT-PCR assays typically take a few hours to perform and often require transporting samples to professional laboratories, which can take a few additional days thus being a challenge during periods of high demand.

COVID-19 and Flu exhibit similar physiological symptoms underscoring the requirement for a rapid diagnostic tool capable of differentially diagnosing COVID-19 and Flu. An initial assessment of the potential cause of illness would allow a timely personalized treatment plan for the patient, thus not only aiding in curbing the spread, but also in utilizing medical resources in an efficient manner. As the recent COVID-19 pandemic spurred the rapid development of multiple COVID-19 detection platforms with varying degrees of usability and success, antibody-modified graphene field effect transistors (GFETs) have stood out due to their low LoDs and fast response time. Imbibing these GFETs with concurrent multiple target detection capability would increase their effectiveness not only during pandemics but also in instances where there is an urgent requirement to detect the cause of illness in a patient showing overlapping symptoms with another disease.

### BRIEF SUMMARY OF THE DISCLOSURE

Accordingly, the present invention is directed to a multi-targeted, modular virus sensing platform based on graphene field-effect sensing that obviates one or more of the problems due to limitations and disadvantages of the related art.

In accordance with the purpose(s) of this invention, as embodied and broadly described herein, this invention, in one aspect, relates to a multi-target specific (e.g., COVID-19 and Influenza) biosensor technology utilizing field-effect transistors (FETs). For example, the FET may be a graphene or other 2D material based FET. The target-specific antibodies or aptamers (whenever available) will be used to add specificity to graphene channels. The technology will enable an early-stage diagnosis tool that differentiates between multiple analytes of choice, and to differentiate between Influenza and COVID-19, yet embodied into a single device for point-of-care monitoring.

In another aspect, the disclosure relates to a biosensor that includes a multiplexed array of electrolyte-gated 2D material based field effect transistors (FET); separating walls on an upper surface of the multiplex array, the walls fluidically separating each of the FETs from one another; and a perimeter wall on the upper surface of the multiplex array surrounding the separating walls and traversing the surface of all FETS in the multiplexed array, the perimeter all forming a fluidically tight well traversing multiple ones of the FETs, the fluidically sealed well subdivided into a plurality of chambers, each chamber corresponding to one of the FETs in the microarray. Each of the FETs includes monolayer of graphene as a conducting channel, wherein each conducting channel functionalized according to a predetermined agent.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

An advantage of the present invention is to provide a rapid response (e.g., within minutes) early-stage diagnosis tool that differentiates between multiple analytes of choice embodied into a single device, which allows for point-of-care monitoring.

Further embodiments, features, and advantages of the multi-targeted, modular virus sensing platform, as well as the structure and operation of the various embodiments of the multi-targeted, modular virus sensing platform, are described in detail below with reference to the accompanying drawings.

It is to be understood that both the foregoing general description and the following detailed description are exemplary and explanatory only and are not restrictive of the invention as claimed.

### BRIEF DESCRIPTION OF THE DRAWINGS

The accompanying figures, which are incorporated herein and form part of the specification, illustrate embodiments of the multi-targeted, modular virus sensing platform. Together with the description, the figures further serve to explain the principles of the multi-targeted, modular virus sensing platform described herein and thereby enable a person skilled in the pertinent art to make and use the multi-targeted, modular virus sensing platform.

FIG. 1 illustrates a schematic of an example biosensor according to principles described herein.

FIGS. 2a, 2b and 2c illustrate preliminary results on the dual [COVID-19 vs Influenza] differentiation via example bi-functionalized GFET array.

FIGS. 3a and 3b illustrate a proposed schematic of a 4-input GFET array.

FIG. 4 further illustrates various processing steps for each of these processes.

FIGS. 5a and 5b show two time traces for two different devices, functionalized with COVID-19 specific antibodies (green), Influenza-specific antibodies (violet), and passivated with tween-20 (red).

FIGS. 6a-6c illustrate characterization results of another dual variant biosensor a device according to principles described herein.

FIGS. 7a-7d refer to simultaneous dual detection of COVID-19 (Spike) and Flu (HA) proteins using a device according to principles described herein.

FIG. 8 illustrates time series specification of a test device against a blank sample and negative control, i.e., BSA.

FIG. 9 is a table illustrating existing antigen/antibody testing platforms compared to this approach.

FIG. 10 shows a benchmark comparing detection of Flu (green) and COVID 19 (orange) according to the present principles with other technologies. The dashed lines represent the minimum limits of detection (LODs) required for different types of samples for successful detection.

FIG. 11 illustrates time response for a device according to the present description upon introduction of both COVID-19 and Flu proteins. (Yellow curve is for COVID-19 and blue curve for Flu).

FIG. 12 illustrates sensitivity of the example device.

FIGS. 13(a)-(f) illustrate the Graphene-based field-effect transistor (GFET) biosensor fabrication process; FIG. 13(g) is a schematic of the final GFET-based biosensor with a polydimethylsiloxane (PDMS) well on top to secure electrolyte;

FIGS. 13(h)-(j) further illustrate graphene functionalization with pyrenebutanoic acid, succinimidyl ester (PASE), an anti-ferritin antibody and the final step of ferritin-specific biosensing.

FIGS. 14(a)-(b) illustrates anti-ferritin antigen binding and functionalization in an example device.

FIG. 15 illustrates change in resistance versus time readings for the example GFET ferritin biosensor on the addition of ferritin-free buffer (phosphate-buffered saline (PBS)) and increasing ferritin concentration.

FIG. 16 is a schematic representation of the dynamic equilibrium of ferritin antigens to immobilized antibody receptors on the active GFET sensor area of an example device.

FIG. 17 shows binding isotherm for the antibody (receptor) occupancy with ferritin antigen on the GFET biosensor of an example device.

### DETAILED DESCRIPTION

Reference will now be made in detail to embodiments of the multi-targeted, modular virus sensing platform with reference to the accompanying figures. The same reference numbers in different drawings may identify the same or similar elements.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

Throughout this application, various publications may have been referenced. The disclosures of these publications in their entireties are hereby incorporated by reference into this application in order to more fully describe the state of the art to which this invention pertains.

Described herein is a multi-target specific (e.g. COVID-19 and Influenza) biosensor technology utilizing graphene-based field-effect transistors (GFETs). The target-specific antibodies or aptamers (whenever available) may be used to add specificity to graphene channels. The technology yields a system that one can use at home, without doctor's or clinician's involvement. For example, a test device according to principles described herein provides a rapid response (e.g., within minutes) and enables an early-stage diagnosis tool that differentiates between multiple analytes of choice, and specifically to differentiate between Influenza and COVID-19, yet embodied into a single device, which allows for point-of-care monitoring.

According to principles described herein an exemplary device uses an FET biosensor having a monolayer graphene as the conducting channel. The channel is functionalized using anti-antibodies or aptamers to selectively capture a bioentity, antigen, or molecule of interest. For example, each individual graphene channels within a device may be functionalized with a different biomolecule. The biomolecules may be specific to bioentities that possess a potential threat to human organism, such as toxins, viruses (COVID-19 and Influenza, Ebola), and other proteins, DNAs, etc. or antigen.

One feature of the technology is multi-specificity and advanced target analyte selectivity. A chip comprising GFETs (or other 2D based FETs) can embody multiple target biomolecule specific counterpads. For example, antibodies or aptamers and provided into the graphene surface. The antibodies or aptamers can be localized within different chambers on the chip, the chambers corresponding to a different GFET channel, making multiple GFETs on a single chip sensitive to different biomolecules of interest. One non-limiting example is a GFET chip having dual biosensing specificity: for COVID-19 and for common Influenza viruses (such as, e.g. N1H1). One chamber may be functionalized as a control. By cross-correlating the response from the functionalized and the control sensors, the device will be able to quickly and decisively give its response. The GFETs may be electrolytic-gated GFETs.

For example, a concurrent rapid differential diagnosis platform using antibody-modified GFET can be fabricated according to principles described herein. The device is a holistic platform having 4 onboard GFETs isolated from each other using polydimethylsiloxane (PDMS) barriers yet enclosed in a higher perimeter PDMS wall so that they can be functionalized individually and tested using a shared biological sample without the assistance of complex microfluidics. Each GFET is modified with either an antibody of interest, i.e., COVID-19 or Flu or are used as a control. The device design enables isolated targeted functionalization of graphene channels while allowing a common medium for introducing the analyte, which then translates into common gating and a change in conductance of the GFET modified with the corresponding target/receptor12. In this case, the chip has two GFETs dedicated to antibody immobilization for COVID-19 and Flu each, while one GFET was only chemically passivated with Tween-20 (Tw20) and another left bare as a control.

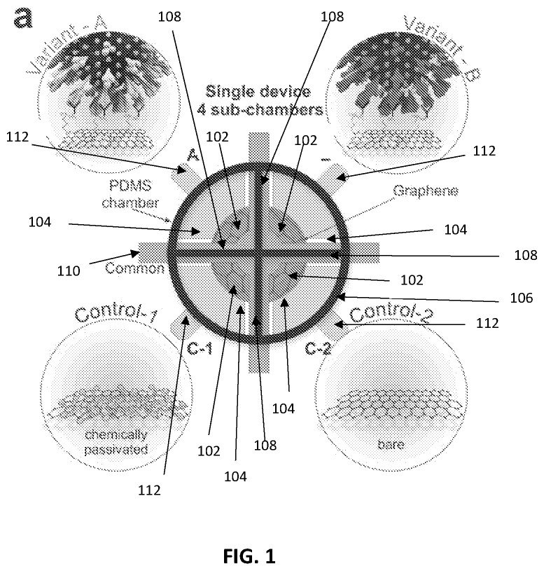

FIG. 1 illustrates a schematic of an example biosensor according to principles described herein. The illustrated biosensor includes four (4) synergistically integrated graphene channels. The channels in the example of FIG. 1 are in a 2×2 matrix, but a chip according to the present disclosure may include any number of channels in any appropriate configuration, including in n×n matrices or n×m matrices, the dimension of which could be determined according to manufacturing or product advantages.

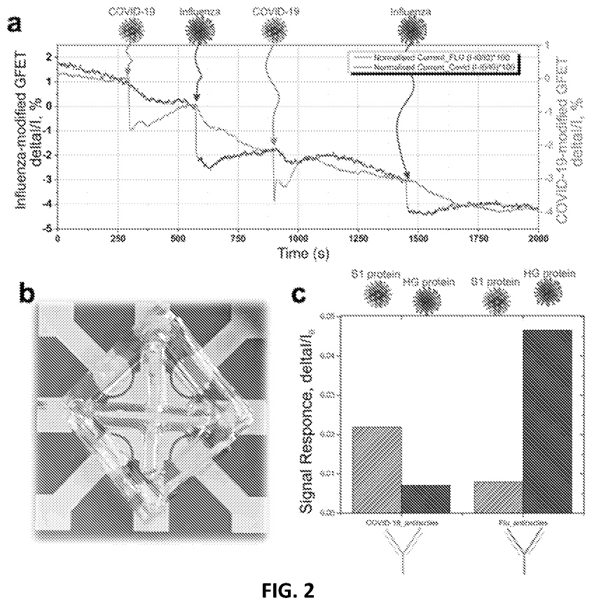

Referring again to FIG. 1, each channel 102 in the example biosensor 100 corresponds to a chamber 104 formed by a perimeter wall 106 and interior separating walls 108 subdividing a well formed by the perimeter wall 106 into multiple chambers 104, each chamber 104 corresponding to one or more GFET channels 102. The device may be configured so that application of a single sample can be used for multiple ones of the chambers. Such a device has a structure where the perimeter wall 106 is higher than the interior separating walls 108 so that an applied fluidic sample can flow over the interior separating walls 108. It is contemplated that the sample has a volume that is held within the well formed by the perimeter wall 106, but such is not required (e.g., no perimeter wall or no concern that the sample overflows). The perimeter wall may be of any shape. For example, the perimeter wall 106 in FIG. 1 has a circular profile, and in FIG. 2 has a square profile. Any perimeter wall path or profile suitable to the array of FETS is possible. For example, in a rectangular array, the path or profile may be rectangular. Regardless, for any array profile, the perimeter wall path or profile may also be circular, oval, triangular, rhomboid, etc., or any other suitable shape, without limitation

In the example illustrated in FIG. 1, the channels are functionalized such that a first channel A is functionalized to a first element, a second channel B is functionalized to a second element B, and the two remaining channels C-1 and C-2 are functionalized as control samples. For example, the first element may be a first Variant A, the second element may be a second Variant B and the controls may be a chemically-passivated control (Control-1) and a bare control (Control-2). This configuration of variants and controls is merely exemplary, and the device may include one or no controls.

The example device illustrated in FIG. 1 includes two control chambers. To test for two biomolecules at the same time, the device includes two chambers in addition to the control chambers. The device is not limited to four chambers, but the device is scalable such that any number of biomolecules can be tested for. For example, a device can include two control chambers plus a number (N) of chambers corresponding to the number of biomolecules to be tested for. As in the device of FIG. 1, a well formed by a perimeter wall can be sub-divided into the appropriate number of functionalized chambers (N) plus two control chambers. Thus, the total number of chambers would be N+2. Of course, more or fewer control chambers may be included in the device without departing from the spirit and scope of this invention.

In an aspect of the device, the device may include a single common electrode 110 to make four chambers (channels), which would use, for example, five leads. For a device with more chambers, the device would use a number of leads equal to the number of chambers (channels) plus one. For example, for a 30 chamber device (28 biosensors plus 2 control), 31 leads would be used. A number of source electrodes 112 may correspond to a number of channels.

Accordingly, an exemplary biosensor includes a multiplexed array of electrolyte-gated graphene based field effect transistors (GFET); separating walls on an upper surface of the multiplex array, the walls fluidically separating each of the GFETs from one another; and a perimeter wall on the upper surface of the multiplex array surrounding the separating walls and traversing the surface of all GFETS in the multiplexed array, the perimeter all forming a fluidically tight well traversing multiple ones of the GFETs, the fluidically sealed well subdivided into a plurality of chambers, each chamber corresponding to one of the GFETs in the microarray. Each of the GFETs includes monolayer of graphene as a conducting channel, wherein each conducting channel functionalized according to a predetermined agent. Examples of functionalizing agents may include DNA, aptamers, antibodies, viral particles (e.g., flu, covid, rotavirus, ebola, zika, etc.), small molecules, peptides, enzymes, bacteria (e.g., salmonella, e-coli, etc.), proteins, exosomes, toxins (e.g., ochratoxin, cholera, etc.), organic molecules (glucose, etc.) or the like.

While described here with a graphene based field effect transistor, other appropriate 2D materials, such as MoS2, hBN, WS2, WSe2, PtS2, PtSe2, could be used with an appropriate electrolyte. The electrolyte may be any one of or a combination of phosphate buffered saline, Hanks' Balanced Salt Solution, Tris, Saliva, nasal swab dissolved in DI water.

The perimeter wall and/or the separating walls may be made of any appropriate material, including a polymer such as polydimethylsilixane, EcoFlex, SU-8, PMMA, a combination thereof or the like. The perimeter wall and/or the separating walls may be SiO2/Si, sapphire, glass, SiC, and flexible polyimides, parylene, polyester, tape, polycarbonate, Teflon, PET, EVA/PET, etc-any substrate which survives chemical treatment with acetone or toluene a combination thereof or the like

The height of the separating walls may be less than height of the perimeter wall. The perimeter wall or the separating walls may be made of silicone, such as polydimethylsilixane. The predetermined agents may include flu antibody, COVID antibody, anti-ferritin antibody or other antigen-specific antibody. As discussed above, at least two of the GFETs can be functionalized differently.

According to the principles described herein, the technology includes:
Electrolyte-gated graphene-based field effect transistor (GFET);
Multiplexed array of multiple GFETs on a single chip;
Ability to functionalize individual graphene channels with different biomolecules that are specific to bioentities that possess a potential threat to human organism, such as toxins, viruses (COVID-19 and Influenza, Ebola), and other proteins, DNAs, etc.

The technological design allows one to (a) functionalize each graphene sub-channel individually, while (b) sample the target analyte with all channels simultaneously. Such design allows us to simplify the overall device fabrication and to bypass the necessity to build microfluidic chambers that would significantly complicate the overall technology and its at-home employment.

Strategy to functionalize the individual graphene channels with individual recognition elements at the same time, without cross-binding. At the same time, all individual channels within the array can be sampled in parallel, and when the target analyte is added into the chamber, it reacts with all individual graphene channels simultaneously.

The technology intends to solve the problem of point-of-care testing in ambulatory, home environments. The final device can be simplified down to a chip that could be ready via a mobile read-out system, and used at home or any place of convenience, utilizing such easy to access bioanalytes as saliva instead of blood.

The technology has the following advantages over current technologies:
1. Response time. Due to the nature of field-effect biosensing and electrical recordings, the device's response time to target analytes is within 10-30 seconds;
2. Superior sensitivity. The currently achieved level of detection is ˜47 ag/mL;
3. Multifunctionality and multi-specificity gives additional potential to avoid false negative results, increasing the overall true positive and true negative percentages.
4. The technology can be modular, when future sensing elements can be developed and later on sort of “lego”-style attached to the existing devices and read-out systems.

Technologically, field-effect biosensing may be restricted with the Debye screening length, which is correlated to the molarity of the solution. The human saline has very high molarity, and if used directly, the Debye layer would be within 1 nm, restricting the ability to electrostatically detect large molecules. This limitation can be overcome by diluting the human saline in deionized water to yield lower molarity and lower concentration bio-analyte.

The technology can be used to detect perhaps any other viral bodies (e.g. ebola or others yet to come) or for detection of hormones, DNAs, proteins, etc.

FIGS. 2a, 2b and 2c illustrate preliminary results on the dual [COVID-19 vs Influenza] differentiation via example bi-functionalized GFET array. FIG. 2a shows time traces of COVID-19 functionalized GFET (red) and Influenza-functionalized GFET (black) as a response to S1 and HG proteins. FIG. 2b is an optical photograph of example bi-functionalized GFET array having the four GFETs in a quadruple array corresponding to the preliminary results of FIG. 2a. FIG. 2c shows cross-reactivity preliminary data.

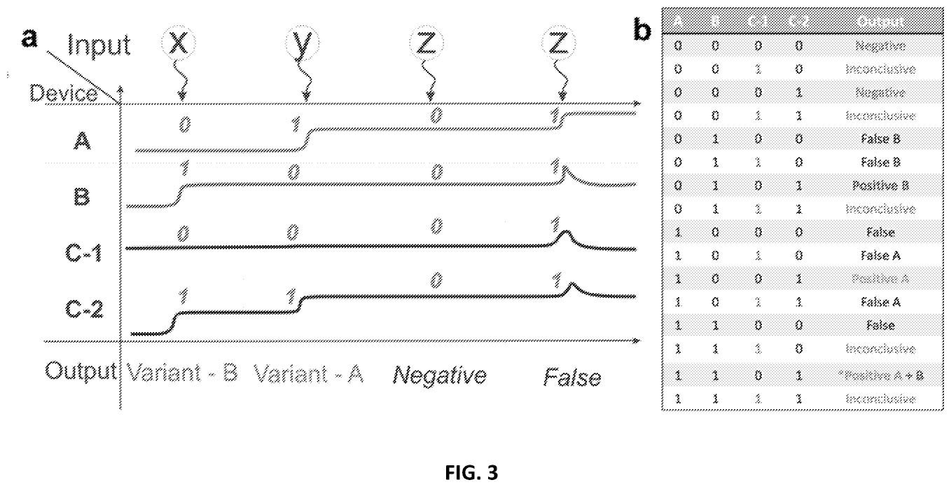

FIGS. 3a and 3b illustrate a proposed schematic of a 4-input GFET array. FIG. 3a shows a proposed schematic of 4-input GFET array (A—functionalized with Element A, B—functionalized with element B, C-1—passivated control device, C-2—bare control device). X, Y, and Z are drops of unknown analyte into the analytical device and based on reading from 4 devices, results can be differentiate and output recognized. FIG. 3b shows is a truth table with 16 possible combinations from such an array.

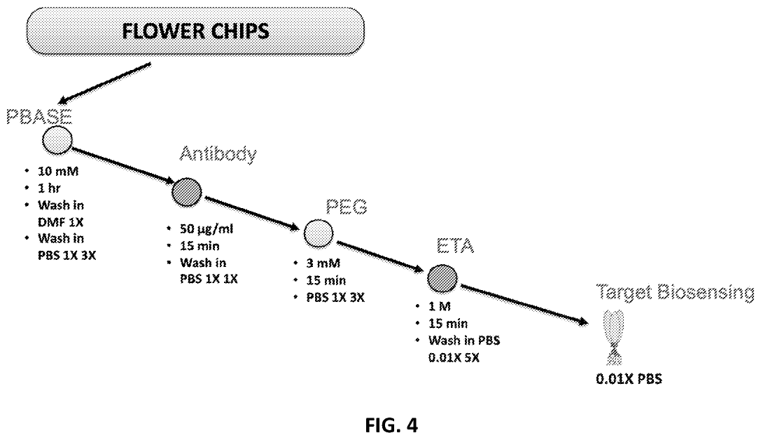

Biosensing according to principles described herein begins with functionalization of the chambers/channels of the GFETs. Referring to FIG. 4, functionalization may include the immobilization of 1-pyrenebutanoic acid succinimidyl ester (PBASE) on top of the graphene channel. The aromatic pyrene groups of PBASE bind very strongly to the graphene surface via a π-π stacking, leaving the succinimidyl ester group at the other end. Then the solution of influenza- or COVID-19 targeted antibodies, dissolved in phosphate buffered solution (PBS), are added onto the graphene surface. The succinimidyl ester groups of PBASE react covalently with the amino groups of the antibody, forming a stable amide bond. After incubation, the GFETs are washed thoroughly with PBS and passivated with amine-terminated Polyethylene Glycol (PEG-NH2) and ethanolamine (ETA). FIG. 4 further illustrates various processing steps for each of these processes.

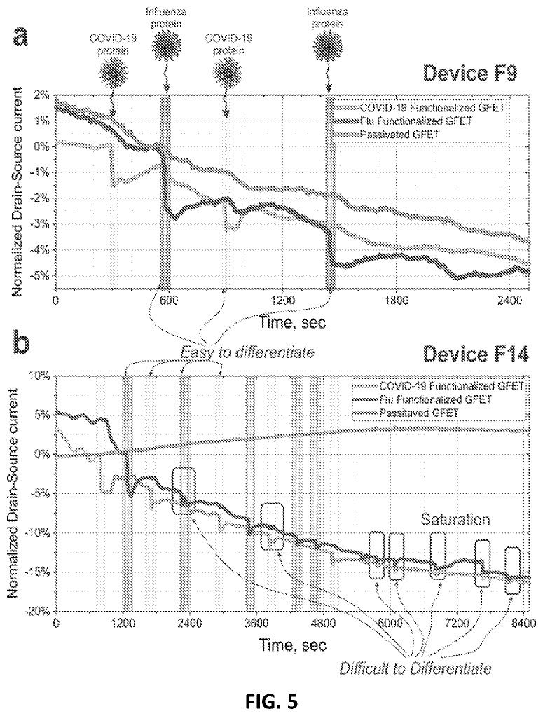

FIG. 5 shows two time traces for two different devices, functionalized with COVID-19 specific antibodies (green), Influenza-specific antibodies (violet), and passivated with tween-20 (red). All GFETs were sampled simultaneously, with the target protein added to the common chamber. The events of COVID-19 biosensing are highlighted in yellow, and events of Influenza are highlighted in light blue.

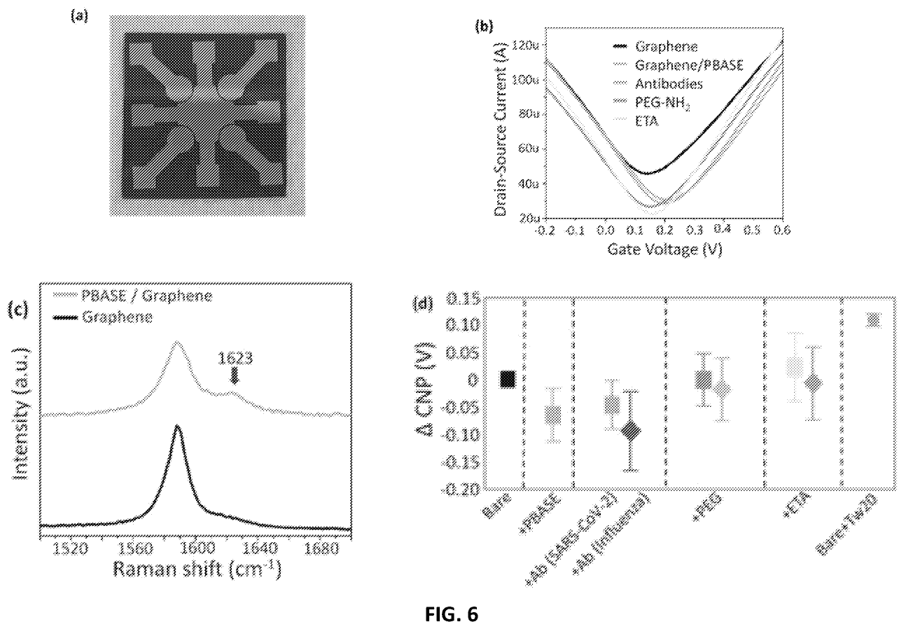

FIGS. 6a-6c illustrate characterization results of another dual variant biosensor a device according to principles described herein. FIG. 6a is an optical image of the sensor with 4 GFETs. FIG. 6b shows transfer curves of the GFETs after each step of functionalization (Black: bare graphene, Yellow Ochre: Graphene with PBASE; Sky blue: Antibodies, Green: PEG-NH2; Yellow: ETA). FIG. 6c illustrates absolute change in Charge Neutrality Point (CNP) at each step of functionalization. Whiskers are ±SD. Samples.

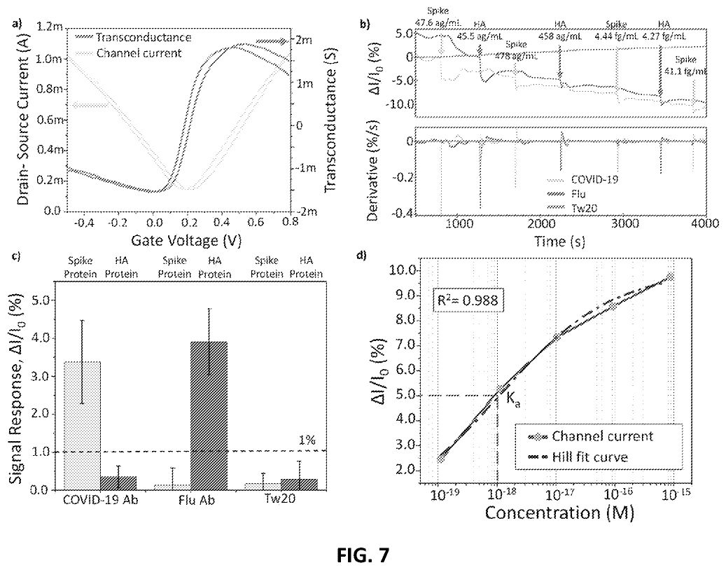

FIGS. 7a-7d refer to simultaneous dual detection of COVID-19 (Spike) and Flu (HA) proteins using a device according to principles described herein. FIG. 7a shows a transconductance curve (vermillion) of the antibody coated GFETs to verify the gate voltage at the point of highest transconductance to ensure the highest sensitivity. FIG. 7b shows time series measurement demonstrating simultaneous detection of both COVID-19 (yellow) and Flu (blue) along with the control (green) and their first derivatives on the same timeline indicating the exact moment of detection and differentiating from other event-induced artifacts. The antigens were introduced in successively increasing concentrations. FIG. 7c shows an average signal response for the interaction with each antibody against Spike and HA across 4 devices at ˜50 ag/mL. A 1% threshold for signal response was assigned to differentiate a specific from a nonspecific antibody binding. FIG. 7d is a Hill-fitted curve of the change in current of GFET immobilized with CR3022 antibodies vs. successively increasing concentration of Spike proteins. Association constant (Ka ˜1×10-18 M) extracted from the Hill-fit curve.

### Example Device

Each device consists of an array of 4 GFETs presenting 4 channels of operation (C-n), isolated from each other through PDMS enclosures (FIG. 1). The ratio of the height of the inner enclosure in the form of a cross with respect to the outer enclosure has been set at 0.6, where the inner enclosure is shorter than the outer enclosure. The height difference between the inner and outer enclosure allows for independent functionalization of each GFET while allowing all the GFETs to be driven through a common gate operating with a common medium during measurements. An Ag/AgCl pellet-based electrode is submerged into the shared medium to act as the gate electrode.

To distinguish between viruses, we selected antibodies that recognize a unique antigen for each virus. For COVID-19, we used the antibody CR3022 to target the receptor-binding domain (RBD) region on the transmembrane Spike protein. For Flu, we selected the engineered antibody FI6v3 to bind to the conserved central stalk domain of transmembrane protein hemagglutinin (HA). The antibodies selected are each capable of binding to multiple variants of their respective virus. For COVID-19, the virus variants haven't mutated to the significance to elicit complete binding escape from antibodies targeting the original virus; thus, most COVID-19 antibodies such as CR3022 are capable of binding to recent variants such as omicron and delta. Additionally, FI6v3 was engineered to bind to all type 1 and 2 influenza A subtypes13. The diversity of variants that can be recognized gives this assay tremendous breadth among different subtypes of each virus. The interaction between the antibodies and their respective analyte proteins was validated through ELISA for each batch of antibodies.

The electric double layer (EDL) formed at the graphene electrolyte interface serves as a dielectric layer. The common electrolyte enabling the operation of the GFETs is a low ionic strength PBS set at 0.01×. The decision to employ PBS 0.01× was to counter the charge screening15-16 effect observed in high ionic concentration solutions, which reduces the observed signal strength17 resulting from the interaction of the target and analyte. It is imperative that EDL fall at the range suitable for IgG antibody interactions, around 4 to 14.5 nm18 as opposed to the low 0.7 nm above the surface EDL formed by PBS 1× 15. Through our experimentation, it was observed that PBS 0.01× served as the best concentration for signal detection while also maintaining bio-molecular integrity as observed through enzyme-linked immunoassay (ELISA).

To allow targeted detection, the GFET channels were modified through biochemical functionalization, starting with making CVD-grown graphene suitable for antibody immobilization. The lack of reactive sites or dangling bonds on CVD graphene offered no site for target immobilization, which was resolved through incubation of 1-pyrenebutanoic acid succinimidyl ester (PBASE) on the surface of graphene. PBASE is a pyrene-based succinimide ester that utilizes the π-π bonds extending out at the surface of graphene. The successful immobilization of PBASE on graphene was confirmed through Raman spectroscopy and electrical characterization. FIG. 6(c) shows the occurrence of a peak at 1623 cm⁻¹ after functionalization of graphene with PBASE, which is concurrent with the presence of pyrene resonance, indicating that PBASE successfully attached to the surface of graphene¹⁹. The reduction of I₂₀/I_{G} ratio from 2.99 to 1.219, from bare to PBASE functionalized graphene, indicates disordered surface further signaling the presence of PBASE, while the rightward shift of the 2D peak by 1.3 cm⁻¹ is indicative of hole doping. Hole doping, being an indicator of PBASE incubation on graphene19, was also confirmed through electrical characterization (FIG. 6(c)-(d)) since the IV curves denote the movement of the charge neutrality point (CNP) rightwards relative to CNP at bare graphene. The CNP at around 0.1V in bare graphene is reflective of doping introduced due to Poly(methyl-methacrylate) (PMMA) residue during the fabrication stage (FIG. 6(d)). The right shift of the CNP indicates the successful stacking of PBASE onto graphene.

The N-hydroxy succinimide (NHS) ester group in PBASE reacts with primary amine groups of the proteins, thus allowing antibody immobilization26. The PDMS enclosure allowed specific immobilization of the CR3022 and FI6v3 onto separate GFETs on the device. To ensure that the area of graphene that remained unoccupied by PBASE and the antibodies did not lead to any non-specific reaction, PEG-NH₂ was introduced as the blocking reagent27. PEG-NH₂ also plays an essential role in combatting the screening effect introduced by the electrolyte, since it increases the Debye length, thus making the EDL comparable to the dimensions of the antibodies28-29. To neutralize PBASE sites unoccupied by antibodies, ETA was used as the blocking agent to prevent any non-specific reaction initiated through the amine groups of analytes being tested. To ensure that the results observed are due to antibody-antigen interaction, rather than electronic drift or fluctuations, we deployed the third GFET as the comparative electronic control. The third graphene channel in this GFET was modified with Tw20 only, to serve as a blocking layer, with the expectation that it would not respond to introduction of any analyte into the solution. Each step of functionalization was characterized electrically and optically with all devices assembled, showing a consistent trend indicating successful immobilization and blocking.

To evaluate the sensing capability of the device, we performed a series of time trace measurements where all onboard transistors were exposed to varying concentrations of both COVID-19 S-protein (Spike) and Flu Hemagglutinin (HA) proteins at different intervals as outlined in the measurement protocol.

The antigen-antibody interaction utilizes the uniform turbulent diffusion of viral proteins delivered in low ionic strength PBS, entailing a facile operating procedure, where the user simply pipettes a drop of the viral protein solution onto the device and observes a response within seconds. Prior to testing the device against the target proteins, a negative control protein test was conducted with bovine serum albumin (BSA) as the analyte to verify its specificity. We established a precise dual detection of the two viruses without cross-reactivity of the signals; hence each time the characterized devices were exposed to control proteins to study cross-reactivity and specificity.

For all the time-resolved trace measurements, the gate voltage was set to the value which exhibited the highest transconductance (Vgmax) for the chip in PBS 0.01× post functionalization (FIG. 7(a)). The gate voltage at the highest transconductance value generally ranged from 120 mV to 200 mV. This was carried out to ensure that the channel had the maximum sensitivity30,32 to any activity on the surface of antibody-decorated graphene channels.

FIG. 7(b) details the response of the quadruple architecture GFET chip to the introduction of both viral surface proteins. The first viral protein to be introduced was Spike protein with the lowest concentration (47.6 ag/mL), following which the channel current stabilized. After stabilization, the second viral surface protein, HA was added with the similar mass concentration as that of the first dosage of Spike protein. For each successive pair of additions, the concentrations of both the control proteins were kept similar. As expected, upon the introduction of Spike protein, the quarter functionalized with CR3022 registered an immediate change in conductance, leading to drop in the current while the GFET functionalized with FI6v3 experienced negligible change. Similarly, the introduction of HA induced a significant drop in channel current in the GFET functionalized with FI6v3 without inciting a significant reaction in the CR3022 GFET, underscoring the high specificity of the functionalization scheme. This can be further confirmed through the change in normalized channel current (ΔI/I0=(I0−I)/I0) observed for the first instance (first concentration at ˜50 ag/mL) of introduction for each protein, as shown in FIG. 2c. The mean change in normalized channel current as observed across the devices tested for COVID-19 GFET upon application of Spike protein is 3.37% (StD: 1%), while upon application of HA is 0.35% (StD: 0.28%). Similarly, upon introducing HA in the GFET with FI6v3, the change in normalized current is 4% (StD: 0.8%), while reaction of Spike protein had a minuscule change of 0.14% (StD: 0.45%). The significant difference in values indicates that the quadruple GFET architecture can successfully identify the control protein while preventing cross reactivity thus demonstrating the capability to function as both a sensitive and specific dual protein detector. The GFET passivated with Tw20 shows a minuscule change in the channel current, averaging at 0.3%, upon addition of any of the above-mentioned analytes, thus serving as a comparative electronic control, revealing the underlying variability in signal without interaction with the biological media. Choosing a cutoff of 1% for the first concentration of the antigens tested we capture 100% of true positives and reject 100% of cross GFET and chemically passivated GFET. 1% change in the normalized signal was chosen as the thresholding value to declare a true positive amongst all the 4 devices since it encompassed the maximum change in normalized current value for cross reactivity observed amongst all the devices (0.9%) while also being 4 times the mean (FIG. 7(c)) response deduced for cross GFET reaction for the first tested concentration.

The derivative of the time series curve eliminates the impact of drift and other electronic artifacts observed in the real-time traces, as shown in FIG. 2b, serving to accurately distinguish the instances of introduction of either Spike or HA protein from other artifacts in the measurements.

As observed in FIG. 7b, response to the first dosage of Spike and HA recorded the most significant drop in source-drain channel current in their respective GFETs in comparison with the successive drops in current observed at later dosages. The amplitude of the change in channel current decreases with an increase in the dosage of the protein. To understand the trend observed in channel current upon addition of successive higher concentrations of protein, kinetics of the antigen-antibody at the graphene interface was examined. The dissociation constant (Kd) is extracted from the ΔI/Io vs. Spike protein concentration Hill-Langmuir model (Eq. 1) fitted protein concentration curve as shown in FIG. 7(d).

$$\frac{Δ I}{Δ {I}_{max}} = \frac{{C}^{n}}{{K}^{n} + {C}^{n}} \qquad ( eq . 1 )$$

The Kd value obtained through the Hill fitted (Eq. 1) data points is 0.147 nM, and the Hill coefficient (n) stands at 0.45. The Hill coefficient below 1 indicates that the interaction between the antigens and the antibodies follows negative cooperative binding33. This implies that the first instance of interaction between the antigen and the antibodies is the strongest, while the reaction at successively increasing concentrations is likely blocked by the presence of viral surface proteins already interacting with antibodies near the surface, leading to a diminished signal response.

When analyzing the device performance, we observed overall sensitivity of the devices is very high, above other emergent technologies. Sensitivity was calculated by performing a linear fit on the linear range of the (I/I0) % vs log (M) curve, achieving 2.4% change in signal per log (molar) concentration for COVID-19 (2.4%/log (M)) and 1.9% change in signal per log (molar) concentration (1.9%/log (M)) of Flu (FIG. 3a). Such sensitivity levels provide superior resolution for detecting and quantifying analytes at extremely low concentrations. Although we report our experimental LoD, practically, the low noise level of our system suggests we could detect down to concentrations of tens of viral surface proteins per mL via single-molecule interactions with the surface42. Apart from the high sensitivity, the devices come with a rapid response time of around ˜10s after addition of analyte (FIG. 3b), which is amongst the fastest response times reported by any platform6. This instantaneous turnaround time, if productized, could be particularly useful in locations with high patient load. Based on the experimental data, the device response's standard deviation (σ) level is 0.04% and 0.07% for COVID-19 and Flu, respectively. Since our lowest detected concentration response is well above the 30 or even 90 value for both COVID-19 and Flu at an average of 3.37% and 4%, respectively, we have an experimental LoD (88 zM) 5-fold lower than the previously reported LoD for detection of COVID-1935. Amongst other technologies like electrochemical sensors40, reporting similar LoDs (Table 1), our device demonstrates the fastest turnaround time while also presenting an inexpensive electronic alternative.

Our device's high sensitivity and low experimental LoD can be attributed to the deployment of low strength ionic buffer and PEG-NH₂ in functionalization to combat the screening effect caused by short Debye length in high ionic strength buffers. Aiding the specific functionalization scheme is also the selection of the most sensitive V_{gs} corresponding to a high transconductance value. By virtue of the linear relationship (Eq. 2) between transconductance and W/L ratio, the high W/L ratio of 8.75 in the device architecture enables higher transconductance, imparting higher sensitivity in turn translating to ultra-low LoD.

$${g}_{m} = ( \frac{W}{L} ) \cdot C \cdot μ \cdot {V}_{DS} \qquad ( eq . 2 )$$

Our device standing at 88 zM is already approaching breath sample detection levels (118.2 zM) while already surpassing the minimum LoD requirements for nasal (163 fM) and saliva sample (16.3 aM). Such low LoD, as exhibited by our device, allows versatility in selecting the type of sample and can potentially reduce the time for administering the test after exposure.

Owing to their molecular weights, theoretically, the lowest possible concentration with Spike and HA protein is ˜1.67 zM. Our device's lowest measured concentrations indicate the capability of almost approaching single molecule detection for each viral protein in their respective GFETs with essentially an immediate turnaround time.

### EXAMPLE METHOD

### ELISA Protocol

High binding 96 well plates [Costar cat 07-200-721] were coated at 2 ug/mL with S protein or HA overnight at 4° C. Plates were washed three times with PBS 1× with 0.05% TW-20 (PBST) and were blocked with PBS 1×, 2% skim milk for 2 hours at room temperature. Antibodies in (1× or 0.01×) PBS, 0.05% TW-20, and 1% skim milk (PBSMT) were serially diluted across the 96 well plate before a 1-hour incubation. Goat Anti-Human-IgG with horseradish peroxidase (HRP) (Sigma-Aldrich™ cat A0293) diluted 1:5000 in PBSMT 1× was used as a secondary antibody and incubated for 30 minutes. 1-Step™ Ultra TMB-ELISA Substrate (Thermo Scientific™ cat 34029) was used to develop the plates and the reaction was quenched with 2M H2S04. Absorbance values were measured at 450 nM on a Synergy H1 microplate reader (BioTek™).

### Proteins

Gblocks ordered from Integrated DNA Technologies (IDT) containing antibody variable heavy or light chains were inserted into mammalian expression vector pcDNA3.4 by Golden Gate cloning and validated with sanger sequencing. Antibodies were expressed using the Expi-293™ Expression System (Thermo Scientific™ cat A14635) and purified with Pierce™ Protein G Plus Agarose (Thermo Scientific™ cat 22851). A stabilized version of the S protein, Hexapro was expressed using the Expi-293 expression system and purified using Ni-NTA agarose (Qiagen cat 30210). All proteins produced in house were validated on SDS-PAGE gels and quantified using the Pierce™ Coomassie Plus (Bradford) Assay Kit (Thermo Scientific™ cat 23236). Proteins purchased commercially included the HA strain H3N2 A/Singapore/INFIMH-16-0019/2016 (Native Antigen) and powdered BSA (Thermo Scientific cat BP9706100).

### Device Preparation

Photolithography and lift off techniques were employed to deposit gold on Si/SiO2 wafer as three terminals to create a 4-GFET array structure of the device. Cr/Au (10 nm/90 nm) layers were deposited through e-beam deposition and lift off techniques. Wet transfer method was utilized to transfer graphene onto the substrate.

Commercially obtained graphene sheet grown on copper (Grolltex) was spin-coated with Poly (methyl methacrylate) (PMMA) (PMMA 950 A4, MicroChem). After spin coating, the PMMA/graphene/Copper stack was baked at 150° C. for 10 minutes. The PMMA/graphene/Copper stack was upturned with the Cu side exposed and was subjected to Oxygen plasma for 30 sec at 30% flow rate. The copper sheet with PMMA/graphene film was then cut into 10 mm×10 mm pieces and placed into Ammonium Persulphate, (NH4)2S2O8, for 24 hours to dissolve the copper. Pieces were placed with PMMA side facing upwards to allow the copper to dissolve. PMMA/graphene film pieces were rinsed and allowed to soak in deionized (DI) water for a total of three consecutive times and then transferred to the silicon wafer with a gold deposit. PMMA/graphene transferred wafers were left to air dry overnight and then baked at 150° C. for 10 minutes. Wafers were then placed in an acetone bath for 24 hours to dissolve the PMMA layer. Bare graphene wafers were rinsed in ethanol and DI water and then dried with the air gun. Dried wafers were baked at 150° C. for 10 minutes. PDMS enclosures were made by cutting rectangular pieces of PDMS and using liquid PDMS to hold them together. The outer PDMS boundary was made with a taller height than the inside cross enclosure to allow overflow between channels on the top (during measurements) of the inside but to prevent leakage to the outside. Inter-leaking between channels was tested using isopropyl alcohol. Small lengths of copper wires were stripped at both ends and connected to the common source, the drain, and the ground through contact with the gold layer on the device and the use of silver epoxy (MG Chemicals 8331S Silver Epoxy Adhesive) to make sure the wires stayed attached to the device.

### Functionalization

10 mM PBASE (Anaspec, AS-81238) solution in Dimethylformamide (DMF) (Thermo Scientific, 20673) was prepared. PBASE and DMF solution was added to both the COVID-19 and Flu-designated GFETs. Glass slide cleaned with ethanol was placed over the device during the 1-hour incubation period to mitigate the risk of DMF evaporating. Starting with one GFET at a time, the PBASE/DMF solution was taken out, and the GFET was rinsed with plain DMF once and DI water three times. Rinsing was performed quickly to avoid drying out the GFET. 50 μg/mL of COVID-19 (CR3022) antibodies were added to the GFET and incubated for an hour. Simultaneously, the Flu-designated GFET went through the same rinsing steps with DMF and DI water with 50 ug/mL of the Flu antibodies, FI6v3, being added with the same incubation time. After one hour of incubation, CR3022 and FI6v3 were taken out of GFET one at a time, and GFET was rinsed with PBS 1× three times. After the rinse, 3 mM PEG-NH2 (Broadpharm, P-22355) and PBS solution were added to the GFET and incubated for another hour. 1M ETA (Sigma Aldrich, 110167) solution was prepared by combining ETA with PBS 1× (pH8). After both GFETs had been incubated with PEG-NH2 for an hour, PEG-NH2 inside the GFET (one GFET at a time) was dispensed and rinsed with PBS 1× three times. The prepared solution of ETA was placed into the GFET and incubated for another hour. All ETA steps were repeated for the other GFET with antibodies. Tw20 (Sigma Aldrich) was placed into a third GFET that didn't contain any antibodies as a negative electronic control. After an hour of incubation with ETA, the ETA solution was dispensed from the GFETs with antibodies and rinsed with PBS 1×. Tw20 was also taken out of its designated GFET, and the GFET was rinsed with PBS 1×.

### Characterization

To ascertain the presence of PBASE and other functionalization reagents on graphene, Raman spectroscopy was performed using Witec Micro-Raman Spectrometer Alpha 300. Electrical functionalization was carried out using Keithley B2909A.

### Device Measurements

Device measurements were carried out using Keysight B2909 A source-meter for both I-V curve and time-resolved measurements. For functionalization step I-V curves, the PDMS chamber was filled with PBS 1×, and the gate voltage was swept over a range of −0.3 to 0.7 V with Vds=0.1V. For time series measurements against the proteins, the PDMS chamber was initially filled with PBS 0.01× at 400 ul and activated with the chosen gate voltage (voltage for highest transconductance) and Vds=0.1V. The chip was allowed to stabilize for at-least 500s. Before introducing the proteins of interest, a third-party test with Bovine Serum Albumin (BSA) was conducted by adding 25 ul of the BSA solution into the PDMS well. After the test, the chip was disconnected from the source meter and thoroughly rinsed and refilled with PBS 0.01× and reconnected to the source meter with the Vgs and Vds set at the same value as previously stated. Once the reconnected chip stabilized, protein samples were introduced at different concentrations. The samples of both Spike and HA proteins were prepared through serial dilution in PBS 1×. Since the buffer being used for testing is PBS 0.01×, the stock proteins prepared in PBS 1× were resuspended in PBS 0.01× (adding 10 ul of protein in 1×PBS into 990 ul of 0.01×PBS) and thoroughly mixed 5 seconds prior to introducing them to the chip (25 uL of the protein in 0.01×PBS added to 400 uL PBS 0.01× solution on the chip). The measurement was performed in pairs, the first 25 ul of Spike protein in PBS 0.01× was introduced into the chip. Once the current stabilized after reaction in the COVID-19 GFET, then 25 ul of HA protein in PBS 0.01× was added to the chip. This procedure was performed for each concentration of protein to be tested.

The proposed invention proves multi-specificity and advanced target analyte selectivity. We propose to embody multiple target biomolecule specific counterpads, often antibodies or aptamers into the graphene surface, making multiple GFETs per chip sensitive to those biomolecules of interest. As an example, a chip that will have a dual biosensing specificity, such as for COVID-19 and for common Influenza H3N2 viruses.

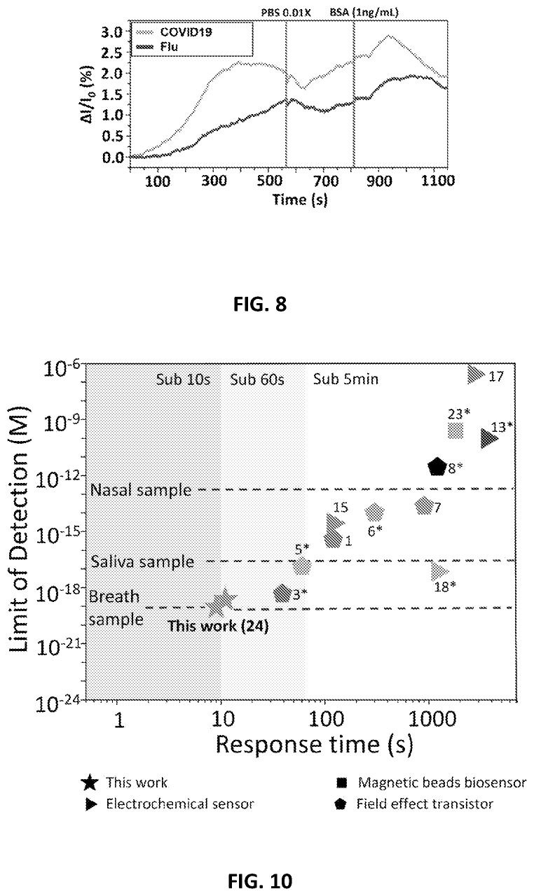

FIG. 8 illustrates time series specification of a test device against a blank sample and negative control, i.e., BSA.

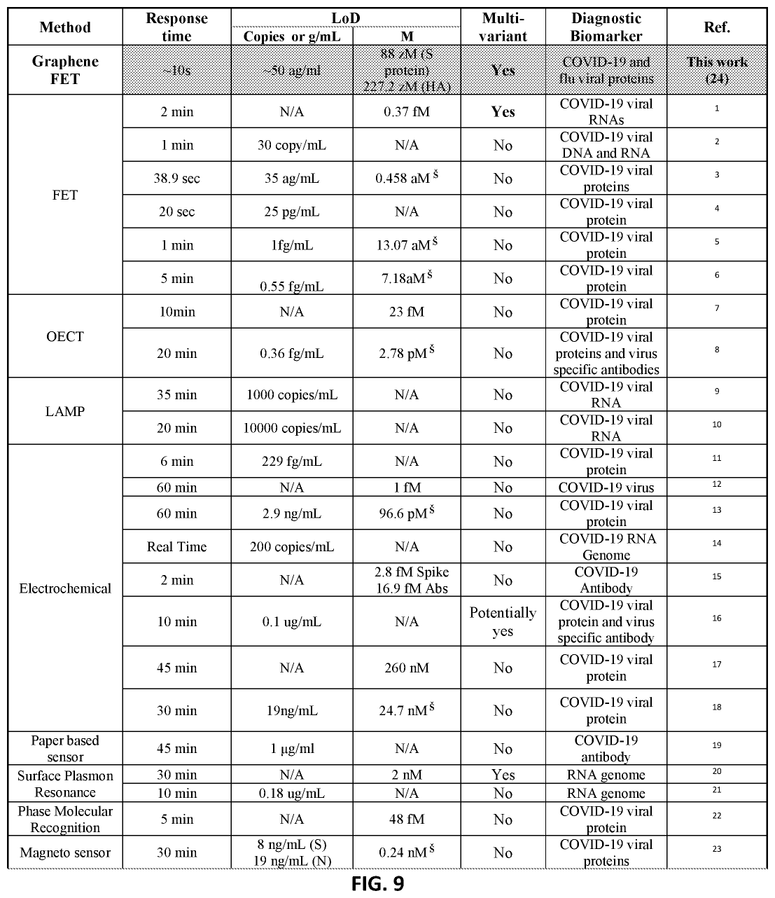

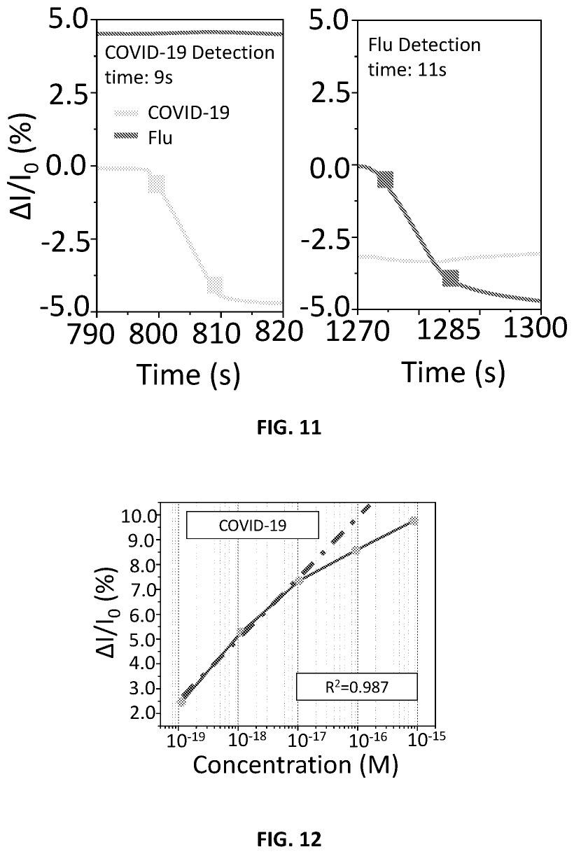

FIG. 9 is a table illustrating existing antigen/antibody testing platforms. FIG. 10 shows a benchmark comparing detection of Flu (green) and COVID 19 (orange) according to the present principles with other technologies. The dashed lines represent the minimum limits of detection (LODs) required for different types of samples for successful detection. FIG. 11 illustrates time response for a device according to the present description upon introduction of both COVID-19 and Flu proteins. (Yellow curve is for COVID-19 and blue curve for Flu). The squares on each curve mark the 10% and the 90% of the step response that occurs due to change in channel current upon interaction of the specific antigen with the antibodies

Due to the nature of field-effect biosensing and electrical recordings, the device's response time to target analytes is within 9-12 seconds. The current sensitivity stands at 2.4% change in signal per log (molar) concentration for COVID-19 (2.4%/log (M)) viral protein particles. 1.9% change in signal per log (molar) concentration (1.9%/log (M)) of Flu. The currently achieved level of detection is ˜47 ag/mL. Multifunctionality and multi-specificity gives additional potential to avoid false negative results, increasing the overall true positive and true negative percentages. The technology can be modular, where future sensing elements can be developed and attached “lego”-style to the existing devices and read-out systems.

One potential limitation is with regards to detection of large biomolecules, such as viruses. Technologically, field-effect biosensing is restricted with the Debye screening length, which is correlated to the molarity of the solution. The human saline has very high molarity, and if used directly, the Debye layer would be within 1 nm, restricting our ability to electrostatically detect large molecules. It can be overcome by diluting the human saline in deionized water to yield lower molarity and lower concentration bio-analyte.

FIG. 12 illustrates sensitivity of the example device. Sensitivity of the GFET for COVID-19 is shown through fitting (blue dotted fitted curve) of the linear part of the concentration vs. current response curve (brown curve with orange data points

Accordingly provided is a multi-target specific (e.g., COVID-19 and Influenza) biosensor technology utilizing graphene-based field-effect transistors (GFETs). The target-specific antibodies or aptamers (whenever available) will be used to add specificity to graphene channels. The technology will enable an early-stage diagnosis tool that differentiates between multiple analytes of choice, and specifically differentiated between Influenza and COVID-19, yet embodied into a single device, for point-of-care monitoring

By cross-correlating the response from the functionalized and the control sensors, the device will be able to quickly and decisively give its response.

Additional Information regarding the certain aspects of experimental results:

The current detected limit of detection is 47 attogram/ml, but this is not the real limit of the system, the device can go at least one order below this limit. The limit is on the order of a few attograms.

Detection range, based on the current experiments varies from a 0.01 mg/ml down to a few ag/ml. The ng/ml is not exactly the ceiling of our recording system. Any concentration above the ceiling will still be detected as “present”.

The specific chip design, large surface area and high-quality monolayer graphene are perhaps the contributing factors for the superior detection limit of the technology.

The technology can be used to detect from real-life samples (e.g., saliva or sweat, with a simple dilution of the samples in DI water).

Unlike the standard RT-PCR testing kits, which have a turn-around time of at least three hours, our device has a faster response time of a few seconds; hence enabling rapid diagnosis. Furthermore, the device is highly specific, as confirmed by cross-reactivity tests: the COVID-19 functionalized channels are not reactive to the Influenza virus, and vice-versa (FIG. 2c). In addition, both channels are not reactive to the introduction of bovine serum albumin (BSA), confirming the outstanding specificity of the multi-target biosensor.

We anticipate (not proven yet) the fabricated biosensors to be able to store for >6 months at 4 C, and >24 hours at room temperature and average (50-100%) humidity.

We have developed design with N=4 compartments. This is a bare minimum to detect and differentiate between two targets. We can increase the number of parallel measurement compartments to any number, e.g., N=12 to differentiate between 10 analytes and N=32 to differentiate between 30 targets.

No special hardware or software has been developed.

Other 2D materials and their heterostructures can be used instead of graphene in the same configuration. MoS2, hBN, WS2, WSe2, PtS2, PtSe2, and others.

Arbitrary substrate can be used. SiO2/Si, sapphire, glass, SiC, and flexible polyimides, parylene, polyester, tape, polycarbonate, Teflon, PET, EVA/PET, etc-any substrate which survives chemical treatment with acetone or toluene.

Detectable items: DNA, aptamers, antibodies, viral particles (flu, covid, rotavirus, ebola, zika, etc.), small molecules, peptides, enzymes, bacteria (e.g., salmonella, e-coli, etc.) proteins, exosomes, toxins (e.g., ochratoxin, cholera, etc.), glucose, etc.

The system can be flipped, and target towards measuring antibodies [ref]

The device is biocompatible, or at least can be made on a biocompatible substrate. The large scale grown graphene alongside the used elements in making the biosensor are biocompatible.

The substrate is insulating, and the electric field over the graphene channel is applied throughout the electrolyte, forming the electrical double layer with high dielectric constant. We commonly use a diluted 0.01×PBS (Phosphate-buffered saline) yet the device can work with any electrolyte (Borate buffered saline, Tris-buffered saline, etc.), even the home tap water.

A single large area (almost around 2×2 cm) large monolayer graphene piece is transferred on top of the pre-fabricated chip with conductive, Au, Pt, or any other metal-based feedlines structured via shadow mask evaporation or photolithography, creating four graphene channels arranged radially with one common electrode and N=4 (or more) radial compartment. After graphene transfer, PDMS chamber with two different heights are placed on top: the inner compartments made of height A, while outer walls with height B. B>A in order to functionalize the compartments separately while measure all for elements for biosensing together at the last stage.

Antibody immobilized GFETs according to principles described herein have registered the lowest measured concentration of the COVID-19 Spike protein and the Flu surface protein, Hemagglutinin (HA), at around 88 zM and 227 zM, respectively. Combined with almost negligible cross-reactivity, we can claim a fast and specific response with the reaction time of ˜10 s depending on the antigen. Together, the performance of the proposed devices opens the possibility of diagnosing patient's conditions well ahead of the 5-day gap suggested by the CDC thus helping in curbing the spread of disease.

Designing for simultaneous and differential detection of COVID-19 and Flu, we describe a sensor platform consisting of an array of GFETs driven through a common gate and shared biological media with LoD at 88 zM for COVID-19 and 227 zM for Flu. These findings provide a proof-of-concept principle solution to the problem of rapidly differentiating two or more diseases with overlapping symptoms. The device enables immediate readout with a rapid turnaround time of around 10s. The differential sensing results from high specificity and sensitivity accorded by the specific immobilization of the antibodies on two GFETs accompanied by an electronic control in the form of passivated GFET. The device presents a highly specific, facile, and portable electronic point of care technology. It would especially benefit areas with high density and volume of patients and visitors such as clinics, nursing homes, universities, offices, etc.; mitigating the bottlenecks created due to high turnaround times and complicated testing procedures presented by conventional technologies. The multi-channel GFET device is also highly versatile since it can be repurposed with antibodies/receptors specific to other diseases, thus serving to track and mitigate future epidemic and pandemic threats.

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative embodiments. Other substitutions, modifications, changes, and omissions may be made in the design, operating conditions, and arrangement of the exemplary embodiments without departing from the scope of the present disclosure.

GFET-based biodetectors have been demonstrated by inventors listed here for the early detection of iron deficiency. These same principles are applicable to functionalizing a GFET for use in a biosensor for any desired antigen by replacing the anti-ferritin antibody with an antibody of choice.

Iron deficiency (ID) is the most prevalent and severe nutritional disorder globally and is the leading cause of iron deficiency anemia (IDA). IDA often progresses subtly symptomatic in children, whereas prolonged deficiency may permanently impair development. Early detection and frequent screening are, therefore, essential to avoid the consequences of IDA. In order to reduce the production cost and complexities involved in building advanced ID sensors, the devices were fabricated using a home-built patterning procedure that was developed and used for this work instead of lithography, which allows for fast prototyping of dimensions.

Described herein is the development of graphene-based field-effect transistors (GFETs) functionalized with anti-ferritin antibodies through a linker molecule (1-pyrenebutanoic acid, succinimidyl ester), to facilitate specific conjugation with ferritin antigen. The resulting biosensors feature an unprecedented ferritin detection limit of 10 fM, indicating a tremendous potential for non-invasive (e.g., saliva) ferritin detection.

Nutrition during the early years of life has a preeminent influence on the quality of health of an individual in their lifetime [1-3]. Specifically, micronutrients provide the essential building blocks for brain development, healthy growth, and a robust immune system [1,4-7]. The top three micronutrients of global health relevance are iodine, iron, and vitamin A, whereas iron deficiency is the most common nutritional disorder worldwide [8,9].

Iron deficiency (ID) refers to a condition of significantly low concentration of healthy red blood cells in the body due to the correspondingly low amount of iron [10,11]. The core function of iron in the body is oxygen transport in the blood. Iron deficiency, if not diagnosed and treated at the early stage, will lead to iron deficiency anemia (IDA). Although every age group is vulnerable, it is more prevalent in women and children [8,12]. However, it is often impossible to recognize ID in children until it degenerates to IDA. At that point, symptoms such as pale skin, frequent infections, fatigue/lethargy, pica, and poor appetite become apparent. ID impairs the cognitive development of children from infancy through to adolescence and is associated with increased morbidity rates [13,14]. It is, therefore, imperative to be able to promptly detect iron deficiencies in children, so that intervention programs are timely and better targeted.

Although iron status is best assessed by a combination of indicators, ferritin is established as the major iron-storage molecule; its production increases in cells as iron supplies increase. The serum ferritin level is, therefore, the most specific biochemical test that correlates with relative total body iron stores; hence, it is the most widely used iron status indicator [8]. However, since ferritin is an acute-phase reactant protein, its concentration is elevated in the presence of infection or inflammation. A child under five years of age is said to be iron-deficient if their serum ferritin level is <12 μg/L, while the threshold is <15 μg/L for children over five years old but rises to <30 μg/L in the presence of an infection [15]. Hence, ferritin tests should be taken very seriously when the results are abnormally low compared to when the measure is normal [10].

Investigating iron deficiency involves a continuous process of recording and assessing iron status in an individual to identify a drop in the indicator levels. Therefore, non-invasiveness becomes necessary, especially when children are involved. The use of saliva presents a non-invasive approach. Saliva is known to contain every information present in the blood but in significantly smaller quantities. Some research works demonstrated the use of saliva for micronutrient testing [16,17]. Moreover, research went into determining salivary ferritin concentrations in humans, as well as correlating serum (or plasma) and salivary ferritin concentrations, as presented in the Table 1, below.

TABLE 1
Correlating serum/plasma and salivary ferritin concentrations
in healthy and iron-deficient (ID) subjects.

| Normal/Healthy (μg/L) | | Iron-Deficient (μg/L) | | |
|---|---|---|---|---|
| Serum/plasma | Saliva | Serum/plasma | Saliva | Reference |
| 196 | 6.5 | — | — | [18] |
| 225 | 948 | 169 | 1114 | [19] |
| 75 | 0.53 | — | — | [20] |
| — | 939 ± 301 | — | 1532 ± 466 | [21] |
| 0.30 ± 0.17 | 0.43 ± 0.42 | 0.067 ± 0.035 | 0.186 ± 0.085 | [22] |
| — | 169 ± 22 | — | Reduced concentration | [23] |
| | | | compared to normal saliva [16] | |

From Table 1, it is clear that the lowest literature-reported iron-deficient salivary ferritin concentration is 0.186 μg/L, which is significantly lower than the 12 μg/L iron-deficient serum ferritin concentration level. The significantly low levels of ferritin in human saliva make it impossible to use the current micronutrient biosensors presented in the literature (presented in Table 2, below).

TABLE 2
Ferritin-targeted micronutrient biosensors.

| Sample | Detection | Performance | | | | | |
|---|---|---|---|---|---|---|---|
| Type | Mechanism | DL | SE | SP | R | RT | Reference |
| Serum | Fluorescence | 250 | — | — | — | — | [24] |
| | | pM | | | | | |
| | Fluorescence | 15 | 88% | 97% | — | 1 [symbol] | [25] |
| | test strip | ng/mL | | | | min | |
| | Photonic crystal | 26 | — | — | Up to | — | [26] |
| | biosensors | mg/mL | | | 2000 ng/mL | | |
| Whole | Lateral flow imm [symbol] | — | 90% | 100% | — | — | [27] |
| blood | (LFIA) | | | | | | |
| | [symbol] PAD to derive plasma; | 5 | 80% | 84% | Up to | 15 | [28] |
| | quantification via light | ng/mL | | | [symbol] 0 ng/mL | min | |
| | transmission changes by | | | | | | |
| | a photodetector | | | | | | |
| PBS | Ho [symbol] -like | 50 | 133.47 | — | Up to | | [29] |
| | silicon [symbol] FET | pg/mL | mV/pH | | 500 ng/mL | | |
| PBS | Graphene FET | 5.3 ng/L | — | — | Up to | 1-10 | This |
| | | (10 [symbol] ) | | | 500 ng/L | s | work |

DL—detection limit; SE—sensitivity; SP—specificity; R—range; RT—response time; PBS—phosphate-buffered saline; FET—field-effect transistor. [symbol]
indicates data missing or illegible when filed

The detection mechanisms of all sensors reported in Table 2, except the silicon nanowire type [29], are all optoelectronic. The biosensor detection range of the lateral flow immunoassay (LFIA)-based sensor is 3-556 μg/L in buffer and 5.78-888 μg/L in serum ferritin standard. However, the sensitivity and specificity reported are based on a detection limit of 18 μg/L. Ferritin concentrations lower than this cut-off resulted in a degradation of the sensitivity and specificity of the biosensor. The silicon nanowire detection mechanism is a departure from the rest in its utilization of a nano-field-effect transistor (FET). This presented the significant advantage of a lower detection limit as compared with others. From an extensive literature search, it was observed that there are significantly few studies on the detection of ferritin concentration using field-effect biosensors. Their method attained a ferritin detection limit down to 50 μg/mL using a horn-like polycrystalline-silicon nanowire (SiNW) FET. Even though their fabrication method is acclaimed to be simpler, the synthesis of silicon nanowires is generally non-trivial and expensive [30,31]. On the other hand, graphene synthesis is simple; graphene is widely commercially available and inexpensive. Moreover, unlike SiNWs, the two-dimensional (2D) planar surface structure of graphene facilitates ease of functionalization. Graphene fabrication and transfer to the substrate are also significantly simple compared to the procedure for SiNWs [32].

Since the first exfoliation of a single atomic layer of graphene in 2004 by Geim and Novoselov [33], of all other nanomaterials, it is known to be the most promising nanostructured material suitable for biosensing, under intense research for over a decade [34,35].

In this research work, we developed an FET biosensor using monolayer graphene as the conducting channel. We functionalized the channel using anti-ferritin antibodies to selectively capture the ferritin protein antigen, with a limit of detection about 10 fM. It is noteworthy that this performance was attained despite using our low-cost and straightforward shadow mask patterning procedure to derive the source and drain electrodes of the graphene-based FETs (GFETs), rather than the standard (ultraviolet (UV) or e-beam) lithography process [36]. This work is the first report of ferritin detection using graphene. It also offers the lowest ferritin detection limit obtainable by any reported sensor. This work demonstrates the enormous potential of using a GFET for non-invasive early detection of iron deficiency.

Graphene on 25-μm-thick copper foil (Gr/Cu) synthesized through chemical vapor deposition (CVD) was purchased from Chongqing Graphene Technology Co., Ltd. (also known as Chongqing Moxi Technology). The following materials were ordered from Millipore Sigma (formerly Sigma-Aldrich): ferritin, anti-ferritin antibody, dimethylformamide (DMF), Tween-20, ethanolamine (ETA), and ˜150 mM phosphate-buffered saline (1×PBS, pH 7.4 at 25° C.). Here, 1.5 mM PBS (0.01×PBS) was prepared by diluting 1×PBS appropriately with de-ionized water. Furthermore, 1-pyrenebutanoic acid, succinimidyl ester (PASE) was purchased from Thermofisher Scientific.

A 285-nm-thick SiO2 on Si wafer was used as a substrate. The source and the drain of the transistor used in this work were patterned according to an interdigitated electrode (IDE). The IDE-structured transistors were fabricated using a shadow mask to pattern the electrodes on top of the SiO2/Si substrate. The masks were fabricated via a simple yet robust technology, which allows for fast prototyping of desirable patterns at a fraction of time and cost, and which utilizes a commercially available, off-the-shelf tool, Silhouette Cameo, capable of providing resolution down to 200 um [37]. A detailed account of this process was reported elsewhere [36]. Although this is a more straightforward approach to patterning in contrast with lithography, there is a limit on the sizes obtainable due to the resolution of the mechanical cutting machine. Each SiO2/Si wafer yielded 28 transistors based on the pre-set dimensions. Since an IDE structure was used, the overall length of the channel was set to 1 mm, and the width was set to 68.8 mm, yielding a ˜69 W/L ratio. Using the CHA e-beam-assisted evaporator, a thin layer each of Ni (10 nm) and Au (90 nm) was deposited.

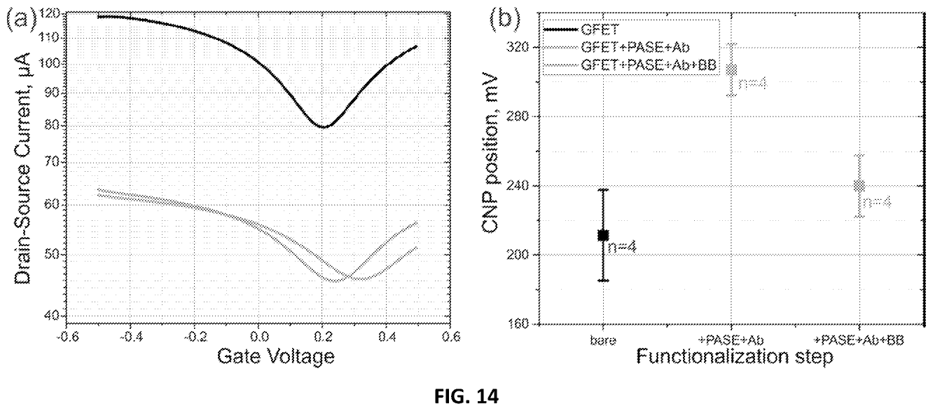

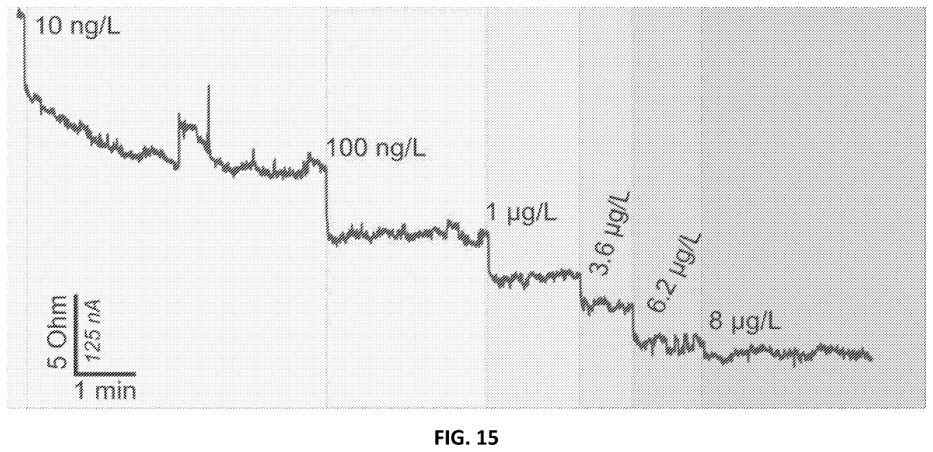

Nickel was deposited first to serve as an adhesion layer, while gold was the metal contact serving as the source and drain for the transistor. The purchased Gr/Cu was cut into desired sizes and stuck onto dummy silicon wafers. A protective polymer (poly(methyl methacrylate) (PMMA)) was drop-casted onto the Gr/Cu and spin-coated for even distribution. The resulting PMMA/Gr/Cu was then annealed at 150° C. for 5 min. This was thereafter transferred onto the etchant (0.1 M ammonium persulfate) to remove the underlying copper foil, leaving PMMA/Gr on top of the solution. The PMMA/Gr was then triple-washed with deionized (DI) water, followed by a careful transfer of a PMMA/Gr sheet onto each IDE-structured transistor to bridge the source and drain electrodes. After the transfer, PMMA/graphene was left to slowly dry out for 12 h at room temperature, followed by 5 min of 150° C. annealing in order to re-flow the PMMA and improve graphene-substrate adhesion. The devices were then left for 24 h in acetone in order to remove the protective PMMA layer, then washed with IPA, and dried with an oxygen gun. Polydimethylsiloxane (PDMS) chambers were then molded and attached to each chip to form an exposed well above the graphene sensing area, thereby creating the means for liquid-based measurements. The GFET fabrication process is summarized in FIGS. 14(a)-(g).

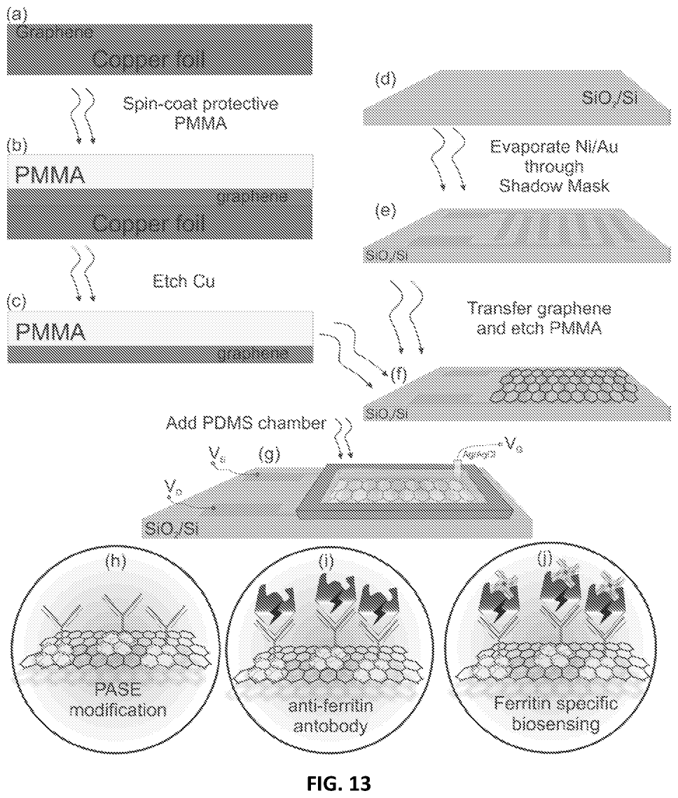

FIGS. 13(a)-(f) illustrate the Graphene-based field-effect transistor (GFET) biosensor fabrication process; FIG. 13(g) is a schematic of the final GFET-based biosensor with a polydimethylsiloxane (PDMS) well on top to secure electrolyte; FIGS. 13(h)-(j) further illustrate graphene functionalization with pyrenebutanoic acid, succinimidyl ester (PASE), an anti-ferritin antibody and the final step of ferritin-specific biosensing.

Transforming a GFET into a specific biosensor requires immobilization of the necessary biomolecules as seen in FIG. 1h-j. To immobilize the biomolecules, a bi-functional linker molecule) 1-pyrenebutanoic acid, succinimidyl ester (PASE) was firstly introduced to the graphene surface. The graphene channel area was incubated in the solution of 10 mM PASE in DMF for 2 h at room temperature. The aromatic pyrene groups of PASE bind very strongly but non-covalently to the graphene surface via π-π stacking, leaving the succinimidyl ester group at the other end (see FIG. 1h). Next, a 1 mg/mL solution of anti-ferritin antibody in 1×PBS was introduced to the GFETs and incubated for 12 h at 4° C. In effect, the succinimidyl ester groups of PASE react covalently with the amino groups of the antibody, forming a stable amide bond (see FIG. 1i). After incubation, the GFETs were washed thoroughly with 1×PBS and DI water, then dried with compressed nitrogen. The graphene surface was then washed with 0.05% Tween-20 in 0.01×PBS to passivate the unbound graphene surface or physically trapped biomolecules. Finally, an additional blocking step of 100 mM ETA in 0.01×PBS was applied to the graphene surface for 1 h at room temperature to deactivate any unreacted succinimidyl ester groups of the PASE that may remain on the surface, followed by thorough washing in 0.01×PBS and DI water, and drying with compressed nitrogen. The GFET functionalization process is summarized in.

After functionalization, the target analyte, ferritin, was prepared in 0.01×PBS to obtain the desired concentrations.

Prior to functionalization, we took the Raman spectra of the graphene on our IDE FET substrate via the Renishaw in Via Raman microscope, using the blue excitation laser wavelength of 442 nm and 4 mW power on the sample to verify the graphene quality and number of layers. The GFETs were electrically characterized at room temperature prior to functionalization, after antibody immobilization and after applying the blocking buffer (Tween-20 and ETA). All measurements were based on a liquid-gated FET set-up. We used 0.01×PBS as the electrolyte buffer solution, and a Keithley B2902A Source Measure Unit (SMU) coupled to a Wentworth Labs probe station. Ag/AgCl pellet electrodes (E-206, Science Products) were used as gate reference electrodes, and they were carefully washed between experiments in order to avoid any cross-contamination. The immobilization processes were characterized by monitoring the drain current changes for a drain-source voltage (VDS) of 0.2 V while sweeping the gate voltage (VGS) from −0.5 to 0.5 V. The sensor performance was determined by monitoring the drain current changes per time for a given drain-source voltage and gate voltage, as the GFET was exposed to the different concentrations of ferritin. The time-trace recordings were performed while keeping both VDS and VGS constant at a certain operational point. The point was set to be VDS=0.1 V and VGS=0.05 V to make sure there were no excessive currents through the graphene.

The used graphene was a high-quality monolayer, as verified by the I2D/IG ratio >2 [38]. The Raman spectrum also revealed a minimal D peak at 1350 cm⁻¹, showing very low defect density. The quality of this graphene facilitated consistent GFET transport properties and confirmed the high fabrication yield of >99% as specified by the manufacturer.

Characterizations were based on transfer curves obtained by plots of drain current versus the gate voltage during stages of fabrication and functionalization of the GFET. The transfer curve obtained by characterizing the bare GFETs immediately after fabrication showed that the GFETs had an average and positive-valued Dirac voltage (Vdirac) of 211±60.4 mV (FIG. 2, black line). This monolayer graphene Dirac point corresponds to the charge neutrality point (with a mean value of Vcnp=211 mV) or the point of least conductivity/maximum resistance. Pristine monolayer graphene (non-doped) has a Vcnp of 0 V; graphene is said to be p-doped when Vcnp is positive and n-doped when Vcnp is negative. Specific anti-ferritin antigen binding was achieved by functionalizing the GFET with an anti-ferritin antibody using PASE as the linker to the graphene surface; this yielded an average Vdirac of 307±33.54 mV (FIG. 14, yellow curve). Usage of the anti-ferritin specific antibody gives us an immense advantage of building a single type of molecule specific biosensor that will be sensitive to ferritin only. Next, a blocking buffer comprising a wash step with 0.05% Tween-20 was applied to remove unbound biomolecules from the graphene surface as much as possible, before incubating the functionalized channel with ETA to block the remaining unreacted N-hydroxy succinimidyl (NHS) ester linkers on the channel surface. Specificity of the biosensor was also assured by means of a previously tested method of additional biosensor passivation performed with ethanolamine and Tween-20 [39,40]. This last functionalization step yielded an average Vdirac of 240±40 mV (FIG. 14, blue line). The decrease in Vdirac was likely due to the removal of weakly bound antibody probes and the nullifying of remnant NHS-ester linker molecules. The standard deviations (SD) of the provided charge neutrality point (CNP) values come from the analysis of multiple (n=4) different GFET chips that were fabricated in a similar manner.

The liquid-gated FET (LG-FET) measurement set-up is the primary measurement configuration for biosensors, where the “liquid” is the sample containing the analyte to be detected or quantified. In this LG-FET set-up, the gate voltage that triggers the modulations in the device is applied to a reference electrode through the liquid to the graphene channel. As this potential is applied, the ELECTRICAL DOUBLE LAYER (EDL) with a capacitance value of CEDL is formed just above the graphene channel. In effect, the CEDL in series with the air-gap capacitance due to graphene's hydrophobicity and the inherent quantum capacitance of graphene produce the total gate capacitance of the GFET. Therefore, a significant advantage of this set-up is the low operating voltage required for the device, typically within 1 V. The thickness of the EDL is a function of the Debye length (λD) as seen in Equation (1). When antigens bind to their antibodies immobilized on the FET surface, a change in surface charge is induced at the binding site. For the changes to be effectively captured, the binding site must be within the Debye length, defined by Equation (2) [41]. Therefore, changes that occur outside this length are subject to electrostatic charge screening.

$$\text{?} = \text{?} / \text{?} \qquad ( 1 )$$

$$\text{?} = \text{?} / \text{?} \qquad ( 2 )$$

$$\text{?} \text{indicates text missing or illegible when filed}$$

where ε0 is the permittivity of free space, εr is the relative permittivity of the dielectric formed between the graphene surface and the liquid, and M (molarity) is the ionic strength of the sample (liquid). from Equation (2), it is evident that a higher molarity results in a shorter Debye length. This concept is of great concern because most biological interactions take place within high-ionic-strength solutions (e.g., 1× PBS ionic strength=˜150 mM). In effect, an attempt to sense these interactions electronically using FET-based sensors is severely impeded by the consequentially short Debye length (0.7 nm for 1×PBS). Therefore, although the binding efficiency of ferritin and its antibody is high due to its large molecular size [42], to ensure this binding is detected by the GFET biosensor, 0.01×PBS (M=1.5 mM, λD=7.3 nm) was used as the electrolyte to carry out the measurements.

The functionalization process incurs some height on the graphene surface that eats into the Debye length. However, the literature highlights that the incurred height from the sensor surface after a flat-on-orientation immobilization of the antibodies is typically about 4 nm [29,43]. Therefore, even for macromolecular antigens like ferritin, using 0.01×PBS will give room for detection of the antigen-antibody binding since the binding site will be within the Debye length of ˜7.3 nm.

For a p-type GFET device, the number of holes is greater than the number of electrons; hence, on the application of the gate voltage, decreased conductivity results. On the other hand, when the GFET is n-type, the application of the gate voltage leads to increased conductivity. However, the immobilization and the binding of charged target biomolecules to receptors on the channel yield specific channel modulation effects. For a p-type device, when a negatively charged biomolecule binds to the receptors on the graphene channel, holes accrue in the channel, leading to increased drain-source current [44]. This binding corresponds to a negative gating potential of the graphene channel and, hence, the reduced carrier density of graphene [45]. On the contrary, when a positively charged biomolecule binds to the receptors on the graphene channel, reduced drain-source current results [46]. Ferritin is a negatively charged molecule with a weight of 474 kDa [47-49]; therefore, with a GFET operated in hole-conduction mode, it is expected that the drain-source current increases (resistance decreases) as the antigen is immobilized on the device. Monitoring of current change is carried out at a certain working potential (0.05 V in this case), and the shift of current is a typical response of biomolecule attachment [40,50-53]. This expected trend can be observed in FIG. 15, which illustrates change in resistance versus time readings for the GFET ferritin biosensor on the addition of ferritin-free buffer (phosphate-buffered saline (PBS)) and increasing ferritin concentration. This figure also represents the points where the highlighted ferritin concentrations pipetted onto the chip resulted in the depicted electrical changes. In this experiment, the ferritin was added onto the chip with initially clean PBS solution. Using simple calculations and a set of four stock ferritin solutions, we gradually increased the concentration of ferritin in the sensing bath, without cleaning or removing the liquid in between. This allowed us to record the gradual change in the response due to the increase in ferritin concentration in a single experiment.

Concerning the detection limit and range of the GFET biosensor, we started pipetting the ferritin antigen onto the chip from the smallest concentration of 10 ng/L, consequently increasing the ferritin concentration up to 8 μg/L. The initial concentration resulted in a significant rise in drain current, which suggests that the smallest analyte concentrations detectable by the developed GFETs are actually lower than 10 ng/L. Notably, the changes in drain current upon ferritin immobilization occurred within less than 10 s of pipetting the protein onto the GFETs, portraying real-time detection.

We consider A (antibody) and F (ferritin) to be two interacting bio-objects which can form a bound product, AF, and we let C_{A}, C_{F}, and C_{AF} be their concentration in M (molarity). The time dependent rate equation for the formation of the product C_{AF} is

$$\frac{\text{?}}{\text{?}} = \text{?} \qquad ( 3 )$$

$$\text{?} \text{indicates text missing or illegible when filed}$$

Where the forward reaction rate constant kₒₙ, and the reverse reaction rate constant k_{off}.

$$\frac{\text{?}}{\text{?}} = - \text{?} \qquad ( 4 )$$

$$\text{?} \text{indicates text missing or illegible when filed}$$

In equilibrium, the sum of all time-dependent derivatives is zero, which in fundamental interpretation obeys the law-of-mass-action equation in solution [54].

$$\text{?} = \frac{\text{?}}{\text{?}} = \frac{\text{?}}{\text{?}} = \frac{\text{?}}{\text{?}} \qquad ( 5 )$$

$$\text{?} = \text{?} \times \text{?} \times \text{?}$$

$$\text{?} \text{indicates text missing or illegible when filed}$$

The strength of the interaction between A (antibody) and F (ferritin) can be linked to the affinity constant Kₐ via the concentration of bound ferritin molecules to the concentration of antibodies. However, it is also necessary to consider the dissociation constant K_{D}, because it can be compared to the reactant ferritin concentrations. In solution, the total concentration of bound antibody-ferritin complex (C_{AF}) depends on the concentration of both antibody and ferritin for biosensors with active surface areas where the law-of-mass-action applies [55].

Immobilized antibodies on the biosensor surface are fixed and, thus, the number of captured ferritin molecules will not change. To have an ideal experiment, the number of ferritin antigens should be in large excess with respect to the number of immobilized antibodies, such that the effective total concentration does not change when ferritin antigens adsorb from the solution to the surface.

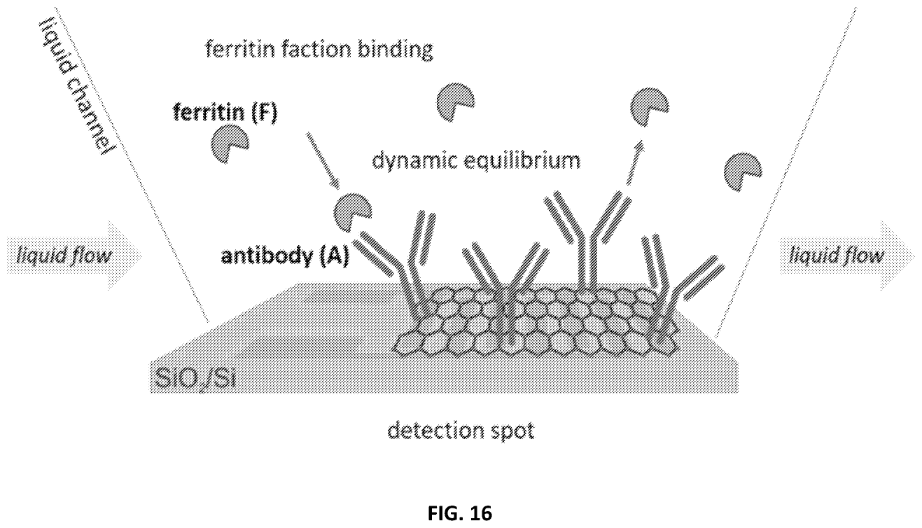

FIG. 16 is a schematic representation of the dynamic equilibrium of ferritin antigens to immobilized antibody receptors on the active GFET sensor area. This simplifies the situation, which, as shown in FIG. 16, is accomplished by providing a constant flow of a fresh analyte solution to the sensor. In a biosensing experiment, an essential quantity is the “bound fraction/ferritin”, Bf value [56], because it is proportional to the measured signal. The bound fraction is defined by the occupied number of ferritin antibodies divided by the total amount of ferritin on the active surface detection area. The antibodies bound are 0% for a ferritin sensor, and they reach 100% when the sensor surface is fully saturated with ferritin analyte molecules.

$$Bound fraction = S \frac{AF}{\text{?}} \text{?} biosensor signal \qquad ( 6 )$$

$$\text{?} \text{indicates text missing or illegible when filed}$$

By combining Equations (5) and (6), we can re-arrange and get the equivalent of the law-of-mass-action for active surface biosensors.

$$Boundfractioninequilibrium , \text{?} ( c ) = \frac{c}{c + \text{?}} \qquad ( 7 )$$

$$\text{?} \text{indicates text missing or illegible when filed}$$

Equation (7) corresponds to the Langmuir isotherm [57], which is derived for the adsorption of the molecules onto surfaces (in this case, on the biosensor surface with attached antibodies) [53,58].

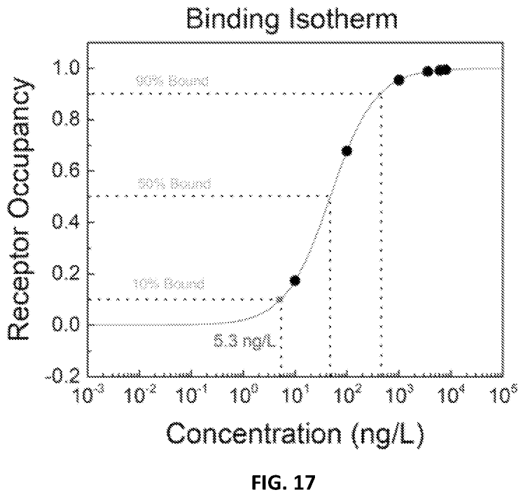

Compared to the law-of-mass-action, this method is simpler and only depends on the ferritin concentration CF and the equilibrium dissociation constant for the antibodies KD. In the specific case of antibodies binding to ferritin antigens, the affinity constant Ka should be calculated. With the known affinity constant, the binding isotherm for the antibody occupancy with the bonded ferritin antigen can easily be plotted (FIG. 17). FIG. 17 shows binding isotherm for the antibody (receptor) occupancy with ferritin antigen on the GFET biosensor. As can be seen from the graph, the lowest ferritin antigen concentration that was successfully bonded to antibodies is equal to 5.3 ng/L (10 fM), which can be indicated as the limit of detection (LoD) for these types of developed.

In this work, we demonstrated the possibility of using graphene to develop an FET biosensor for the detection of serum ferritin protein, whose level gives reliable information about iron deficiencies in the human body. This is the first reported GFET biosensor for ferritin detection. These GFETs were fabricated using our innovative and low-cost method of preparing a shadow mask for patterning and evaporating metal contacts on the substrate. From our analysis, the ferritin detection limit of the GFET biosensor is 5.3 ng/L (10 fM), which is the lowest detection limit reported for ferritin in the literature, while the detection range is 5.3 ng/L (10 fM) to ˜0.5 μg/L (1 pM). These results show that there is excellent potential in using these GFETs for non-invasive ferritin sensing characterized by very low detection limits.

References discussed in paragraphs [0074]-[00108] are as follows:
1. Prado, E. L.; Dewey, K. G. Nutrition and brain development in early life. Nutr. Rev. 2014, 72, 267-284. [CrossRef] [PubMed]
2. UNICEF. Nutrition in the First 1000 Days; UNICEF: New York, NY, USA, 2012.
3. Oshin, O.; Hampel, D.; Idachaba, F.; Atayero, A. The First 1000 Days: Trends towards Biosensing in Assessing Micronutrient Deficiencies. J. Phys. Conf. Ser. 2019, 1299, 012136. [CrossRef]
4. Prado, E. L.; Sebayang, S. K.; Apriatni, M.; Adawiyah, S. R.; Hidayati, N.; Islamiyah, A.; Siddiq, S.; Harefa, B.; Lum, J.; Alcock, K. J.; et al. Maternal multiple micronutrient supplementation and other biomedical and socioenvironmental influences on children's cognition at age 9-12 years in Indonesia: Follow-up of the SUMMIT randomised trial. Lancet Glob. Health 2017, 5, e217-e228. [CrossRef]
5. Baker, R.; Caballero, B. H.; Dennison, B. A.; Innis, S. M.; Simmons, R.; Bonny, S. Toddler Nutrition. Mead Johnson Nutritionals. 2007. Available online: https://www.meadjohnson.com/pediatrics/us-en/sites/hcp-usa/files/LB2264.pdf (accessed on 11 Mar. 2020).
6. Shergill-Bonner, R. Micronutrients. Paediatr. Child Health 2013, 23, 331-336. [CrossRef]
7. Bailey, R. L.; West, K. P., Jr.; Black, R. E. The Epidemiology of Global Micronutrient Deficiencies. Ann. Nutr. Metab. 2015, 66, 22-33. [CrossRef] [PubMed]
8. WHO. Evaluating the public health significance of micronutrient malnutrition. In Guidelines on Food Fortification with Micronutrients; WHO: Geneva, Switzerland, 2010; pp. 41-92.
9. Lopez, A.; Cacoub, P.; Macdougall, I. C.; Peyrin-Biroulet, L. Iron deficiency anaemia. Lancet 2015, 387, 907-916. [CrossRef]
10. Lu, S.-Y. Perception of iron deficiency from oral mucosa alterations that show a high prevalence of Candida infection. J. Formos. Med. Assoc. 2016, 115, 619-627. [CrossRef]
11. Lynch, S.; Pfeier, C. M.; Georgie, M. K.; Brittenham, G.; Fairweather-Tait, S.; Hurrell, R. F.; McArdle, H. J.; Raiten, D. J. Biomarkers of Nutrition for Development (BOND)-Iron Review. J. Nutr. 2018, 148, 1001S-1067S. [CrossRef]
12. Freerks, L.; Papadatou Soulou, E.; Batchelor, H.; Klein, S. A review of GI conditions critical to oral drug absorption in malnourished children. Eur. J. Pharm. Biopharm. 2019, 137, 9-22. [CrossRef]
13. Kuku-Shittu, O.; Onabanjo, O.; Fadare, O.; Oyeyemi, M. Child Malnutrition in Nigeria: Evidence from Kwara State; International Food Policy Research Institute: Washington, DC, USA, 2016.
14. Lozoff, B. Early Iron Deficiency Has Brain and Behavior Effects Consistent with Dopaminergic Dysfunction. J. Nutr. 2011, 141, 740S-746S. [CrossRef] [PubMed]
15. WHO; UNICEF; U N U. Iron Deficiency Anaemia: Assessment, Prevention and Control; WHO: Geneva, Switzerland, 2001.
16. Höller, U.; Bakker, S. J. L.; Düsterloh, A.; Frei, B.; Köhrle, J.; Konz, T.; Lietz, G.; McCann, A.; Michels, A. J.; Molloy, A. M.; et al. Micronutrient status assessment in humans: Current methods of analysis and future trends. TrAC Trends Anal. Chem. 2018, 102, 110-122. [CrossRef]
17. Kanneppady, S. K.; Bhaskar, A.; Kanneppady, S. S.; Rao, P. K.; Ramasamy, S.; Lakshman, A. R.; Shetty, S. R.; Devi, U. H.; Oo, A. M.; Lwin, O. M. Analysis of Salivary Micronutrients in Oral Potentially Malignant Disorder. J. Nutr. Health Food Sci. 2018, 6, 1-4. [CrossRef]
18. Guo, L.-N.; Yang, Y.-Z.; Feng, Y.-Z. Serum and salivary ferritin and Hepcidin levels in patients with chronic periodontitis and type 2 diabetes mellitus. BMC Oral Health 2018, 18, 63. [CrossRef]
19. Aghazadeh, M.; Eslami, H.; Pirzadeh, A.; Aghazadeh, Z.; Dousti, H.; Falsafi, P. Evaluation of Correlation Between Saliva And Serum Ferreting Level In Patients With Iron Deficiency Anemia And Comparison Group. IOSR J. Humanit. Soc. Sci. (IOSR-JHSS) 2016, 21, 54-59.
20. Khan, A. Detection and quantitation of forty eight cytokines, chemokines, growth factors and nine acute phase proteins in healthy human plasma, saliva and urine. J. Proteom. 2012, 75, 4802-4819. [CrossRef]
21. Jagannathan, N.; Thiruvengadam, C.; Ramani, P.; Premkumar, P.; Natesan, A.; Sherlin, H. J. Salivary ferritin as a predictive marker of iron deficiency anemia in children. J. Clin. Pediatr. Dent. 2012, 37, 25-30. [CrossRef]
22. Canatan, D.; Akdeniz, S. K. Iron and ferritin levels in saliva of patients with Thalassemia and iron deficiency anemia. Mediterr. J. Hematol. Infect. Dis. 2012, 4, e2012051. [CrossRef]
23. Agarwal, P. K.; Agarwal, K. N.; Agarwal, D. K. Biochemical changes in saliva of malnourished children. Am. J. Clin. Nutr. 1984, 39, 181-184. [CrossRef]
24. Kartalov, E. P.; Lin, D. H.; Lee, D. T.; Anderson, W. F.; Taylor, C. R.; Scherer, A. Internally calibrated quantification of protein analytes in human serum by fluorescence immunoassays in disposable elastomeric microfluidic devices. Electrophoresis 2008, 29, 5010-5016. [CrossRef]
25. Lu, Z.; O'Dell, D.; Srinivasan, B.; Rey, E.; Wang, R.; Vemulapati, S.; Mehta, S.; Erickson, D. Rapid diagnostic testing platform for iron and vitamin A. deficiency. Proc. Natl. Acad. Sci. USA 2017, 114, 13513-13518. [CrossRef] [PubMed]
26. Peterson, R. D.; Cunningham, B. T.; Andrade, J. E. A photonic crystal biosensor assay for ferritin utilizing iron-oxide nanoparticles. Biosens. Bioelectron. 2014, 56, 320-327. [CrossRef] [PubMed]
27. Srinivasan, B.; O'Dell, D.; Finkelstein, J. L.; Lee, S.; Erickson, D.; Mehta, S. ironPhone: Mobile device-coupled point-of-care diagnostics for assessment of iron status by quantification of serum ferritin. Biosens. Bioelectron. 2018, 99, 115-121. [CrossRef] [PubMed]
28. Lee, S.; Aranyosi, A. J.; Wong, M. D.; Hong, J. H.; Lowe, J.; Chan, C.; Garlock, D.; Shaw, S.; Beattie, P. D.; Kratochvil, Z. F.; et al. lexible opto-electronics enabled microfluidics systems with cloud connectivity for point-of-care micronutrient analysis. Biosens. Bioelectron. 2016, 78, 290-299. [CrossRef] [PubMed]
29. Yen, L.-C.; Pan, T.-M.; Lee, C.-H.; Chao, T.-S. Label-free and real-time detection of ferritin using a horn-like polycrystalline-silicon nanowire field-effect transistor biosensor. Sens. Actuators B Chem. 2016, 230, 398-404. [CrossRef]
30. Namdari, P.; Daraee, H.; Eatemadi, A. Recent Advances in Silicon Nanowire Biosensors: Synthesis Methods, Properties, and Applications. Nanoscale Res. Lett. 2016, 11, 406. [CrossRef]
31. Nair, P. R.; Alam, M. A. Design Considerations of Silicon Nanowire Biosensors. IEEE Trans. Electron. Devices 2007, 54, 3400-3408. [CrossRef]
32. Ambhorkar, P.; Wang, Z.; Ko, H.; Lee, S.; Koo, K. I.; Kim, K.; Cho, D. D. Nanowire-Based Biosensors: From Growth to Applications. Micromachines 2018, 9, 679. [CrossRef]
33. Novoselov, K. S.; Geim, A. K.; Morozov, S. V.; Jiang, D.; Zhang, Y.; Dubonos, S. V.; Grigorieva, I. V.; Firsov, A. A. Electric field effect in atomically thin carbon films. Science 2004, 306, 666-669. [CrossRef]
34. Chauhan, N.; Maekawa, T.; Kumar, D. N. S. Graphene based biosensors—Accelerating medical diagnostics to new-dimensions. J. Mater. Res. 2017, 32, 2860-2882. [CrossRef]
35. Peña-Bahamonde, J.; Nguyen, H. N.; Fanourakis, S. K.; Rodrigues, D. F. Recent advances in graphene-based biosensor technology with applications in life sciences. J. Nanobiotechnol. 2018, 16, 75. [CrossRef] [PubMed]
36. Oshin, O.; Kireev, D.; Akinwande, D.; Adetiba, E.; Idachaba, F.; Atayero, A. Advancing PoC Devices for Early Disease Detection using Graphene-based Sensors. J. Phys. Conf. Ser. 2019, 1378, 032031. [CrossRef]
37. Yang, S.; Chen, Y. C.; Nicolini, L.; Pasupathy, P.; Sacks, J.; Su, B.; Yang, R.; Sanchez, D.; Chang, Y. F.; Wang, P.; et al. ‘Cut-and-Paste’ Manufacture of Multiparametric Epidermal Sensor Systems. Adv. Mater. 2015, 27, 6423-6430. [CrossRef] [PubMed]
38. Li, X.; Cai, W.; An, J.; Kim, S.; Nah, J.; Yang, D.; Piner1, R.; Velamakanni, A.; Jung, I.; Tutuc, E.; et al. Large-area synthesis of high-quality and uniform graphene films on copper foils. Science 2009, 324, 1312-1314. [CrossRef] [PubMed]
39. Zhang, X.; Jing, Q.; Ao, S.; Schneider, G. F.; Kireev, D.; Zhang, Z.; Fu, W. Ultrasensitive Field-Effect Biosensors Enabled by the Unique Electronic Properties of Graphene. In Small; Wiley-VCH Verlag: Weinheim, Germany, 2019; p. 1902820.
40. Fu, W.; Feng, L.; Panaitov, G.; Kireev, D.; Mayer, D.; Offenhäusser, A.; Krause, H. Biosensing near the neutrality point of graphene. Sci. Adv. 2017, 3, e1701247. [CrossRef] [PubMed]
41. Fu, W.; Jiang, L.; van Geest, E. P.; Lima, L. M. C.; Schneider, G. F. Sensing at the Surface of Graphene Field-Effect Transistors. Adv. Mater. 2017, 29. [CrossRef]
42. Ikai, A. The World of Nano-Biomechanics: Mechanical Imaging and Measurement by Atomic Force Microscopy; Elsevier: Amsterdam, The Netherlands, 2008.
43. Hideshima, S.; Sato, R.; Kuroiwa, S.; Osaka, T. Fabrication of stable antibody-modified field effect transistors using electrical activation of Schiff base cross-linkages for tumor marker detection. Biosens. Bioelectron. 2011, 26, 2419-2425. [CrossRef]
44. Kazuhiko, M.; Yasuhide, O.; Kenzo, M. Utilizing research into electrical double layers as a basis for the development of label-free biosensors based on nanomaterial transistors. Nanobiosens. Dis. Diagnosis 2015, 5, 1-13.
45. Teixeira, S.; Burwell, G.; Castaing, A.; Gonzalez, D.; Conlan, R. S.; Guy, O. J. Epitaxial graphene immunosensor for human chorionic gonadotropin. Sens. Actuators B Chem. 2014, 190, 723-729. [CrossRef]
46. Mao, S.; Lu, G.; Yu, K.; Bo, Z.; Chen, J. Specific protein detection using thermally reduced graphene oxide sheet decorated with gold nanoparticle-antibody conjugates. Adv. Mater. 2010, 22, 3521-3526. [CrossRef]
47. He, D.; Marles-Wright, J. Ferritin family proteins and their use in bionanotechnology. New Biotechnol. 2015, 32, 651-657. [CrossRef]
48. Uto, K.; Yamamoto, K.; Kishimoto, N.; Muraoka, M.; Aoyagi, T.; Yamashita, I. Electrostatic adsorption of ferritin, proteins and nanoparticle conjugate onto the surface of polyelectrolyte multilayers y. J. Mater. Chem. 2008, 18, 3876-3884. [CrossRef]
49. Takahashi, T.; Kuyucak, S. Functional properties of threefold and fourfold channels in ferritin deduced from electrostatic calculations. Biophys. J. 2003, 84, 2256-2263. [CrossRef]
50. Huang, Y.; Dong, X.; Liu, Y.; Li, L. J.; Chen, P. Graphene-based biosensors for detection of bacteria and their metabolic activities. J. Mater. Chem. 2011, 21, 12358-12362. [CrossRef]
51. Ohno, Y.; Maehashi, K.; Matsumoto, K. Label-free biosensors based on aptamer-modified graphene field-effect transistors. J. Am. Chem. Soc. 2010, 132, 18012-18013. [CrossRef]
52. Maehashi, K.; Katsura, T.; Kerman, K.; Takamura, Y.; Matsumoto, K.; Tamiya, E. Label-free protein biosensor based on aptamer-modified carbon nanotube field-effect transistors. Anal. Chem. 2007, 79, 782-787. [CrossRef]
53. Saltzgaber, G.; Wojcik, P.; Sharf, T.; Leyden, M. R.; Wardini, J. L.; Heist, C. A.; Adenuga, A. A.; Remcho, V. T.; Minot, E. D. Scalable graphene field-effect sensors for specific protein detection. Nanotechnology 2013, 24, 355502. [CrossRef]
54. Schuster, P.; Érdi, P.; Tóth, J. Mathematical Models of Chemical Reactions. Theory and Applications of Deterministic and Stochastic models. A volume in the series: Nonlinear Science, Theory and Application. Manchester University Press, Manchester (U.K.) 1989. 259 Seiten. Berichte der Bunsengesellschaft für Phys. Chem. 1989, 93, 1501-1502. [CrossRef]
55. Hammond, J. L.; Formisano, N.; Estrela, P.; Carrara, S.; Tkac, J. Electrochemical biosensors and nanobiosensors. Essays Biochem. 2016, 60, 69-80.
56. Pollard, T. D. MBOC technical perspective: A guide to simple and informative binding assays. Mol. Biol. Cell 2010, 21, 4061-4067. [CrossRef]
57. Swenson, H.; Stadie, N. P. Langmuir's Theory of Adsorption: A Centennial Review. Langmuir 2019, 35, 5409-5426. [CrossRef] [PubMed]
58. Li, Y.; Wang, C.; Zhu, Y.; Zhou, X.; Xiang, Y.; He, M.; Zeng, S. Fully integrated graphene electronic biosensor for label-free detection of lead (II) ion based on G-quadruplex structure-switching. Biosens. Bioelectron. 2017, 89 Pt 2, 758-763. [CrossRef]

While various embodiments of the present invention have been described above, it should be understood that they have been presented by way of example only, and not limitation. It will be apparent to persons skilled in the relevant art that various changes in form and detail can be made therein without departing from the spirit and scope of the present invention. Thus, the breadth and scope of the present invention should not be limited by any of the above-described exemplary embodiments, but should be defined only in accordance with the following claims and their equivalents.

1. A biosensor comprising:
a multiplexed array of electrolyte-gated field effect transistors (FET);
separating walls on an upper surface of the multiplexed array, the walls fluidically separating each of the FETs from one another; and
a perimeter wall on the upper surface of the multiplexed array surrounding the separating walls and traversing the surface of all FETS in the multiplexed array, the perimeter wall forming a fluidically tight well traversing multiple ones of the FETs, the fluidically sealed well subdivided into a plurality of chambers, each chamber corresponding to one of the FETs in the multiplexed array;
wherein each FET comprises monolayer of a two dimensional (2D) material as a conducting channel, wherein each conducting channel functionalized according to a predetermined agent.

2. The biosensor of claim 1, wherein height of the separating walls is less than height of the perimeter wall.

3. The biosensor of claim 1, further comprising an electrolyte in the well.

4. The biosensor of claim 3, wherein the electrolyte is one of or a combination of phosphate buffered saline, Hanks' Balanced Salt Solution, Tris, Saliva, nasal swab dissolved in DI water or the like.

5. The biosensor of claim 1, wherein the separating walls comprise a polymer.

6. The biosensor of claim 1, wherein the separating walls comprise polydimethylsilixane, EcoFlex, SU-8, PMMA, a combination thereof or the like.

7. The biosensor of claim 1, wherein the separating walls comprise SiO2/Si, sapphire, glass, SiC, and flexible polyimides, parylene, polyester, tape, polycarbonate, Teflon, PET, EVA/PET.

8. The biosensor of claim 1, wherein the perimeter wall comprises a polymer.

9. The biosensor of any one of claim 1, wherein the perimeter wall comprises polydimethylsilixane, EcoFlex, SU-8, PMMA, a combination thereof or the like.

10. The biosensor of claim 1, wherein the perimeter wall comprises SiO2/Si, sapphire, glass, SiC, and flexible polyimides, parylene, polyester, tape, polycarbonate, Teflon, PET, EVA/PET, or the like.

11. The biosensor of claim 1, wherein at least one of the predetermined agents is a control.

12. The biosensor of claim 1, wherein at least one of the predetermined agents is flu antibody.

13. The biosensor of claim 1, wherein at least one of the predetermined agents is a COVID antibody.

14. The biosensor of claim 1, wherein at least one of the predetermined agents is an anti-ferritin antibody.

15. The biosensor of claim 1, wherein at least two of the FETs are functionalized differently.

16. The biosensor of claim 1, wherein the multiplexed array comprises an n×m array of FETs.

17. The biosensor of claim 14, wherein n=m.

18. The biosensor of claim 1, wherein the multiplexed array comprises 4 FETs in a 2×2 array.

19. The biosensor of claim 1, wherein the multiplexed array comprises 9 FETs in a 3×3 array.

20. The biosensor of claim 1, wherein the 2D material includes graphene, MoS2, hBN, WS2, WSe2, PtS2, and/or PtSe2, a combination thereof or the like.

21-27. (canceled)
